SEQUENCE LISTING (39 sequences)

SEQ ID NO: 1 - protein, 127 aa, Rana pipiens
MFPKFSFLLIFAVVLSLTHKSLCQDWLTFQKKHITNTRDVDCDNIMSTNLFHCKDKNTFIYSRPEPVKAICKGIIASKNVLTTSEFYLSDCNVTSRPCKYKLKKSTNKFCVTCENQAPVHFVGVGSC

SEQ ID NO: 2 - protein, 104 aa, Rana pipiens
QDWLTFQKKHITNTRDVDCDNIMSTNLFHCKDKNTFIYSRPEPVKAICKGIIASKNVLTTSEFYLSDCNVTSRPCKYKLKKSTNKFCVTCENQAPVHFVGVGSC

SEQ ID NO: 3 - protein, 104 aa, Artificial Sequence
  Recombinant Ranpirnase variant
EDWLTFQKKHITNTRDVDCDNILSTNLFHCKDKNTFIYSRPEPVKAICKGIIASKNVLTTSEFYLSDCNVTSRPCKYKLKKSTNKFCVTCENQAPVHFVGVGSC

SEQ ID NO: 4 - protein, 104 aa, Artificial Sequence
  Ranpirnase variant
EDWLTFQKKHVTNTRDVDCNNILSTNLFHCKDKNTFIYSRPEPVKAICKGIIASKNVLTTSEFYLSDCNVTSRPCKYKLKKSTNKFCVTCENQAPVHFVGVGRC

SEQ ID NO: 5 - protein, 104 aa, Artificial Sequence
  Recombinant Ranpirnase variant
QDWLTFQKKHITNTRDVDCDNILSTNLFHCKDKNTFIYSRPEPVKAICKGIIASKNVLTTSEFYLSDCNVTCRPCKYKLKKSTNKFCVTCENQAPVHFVGVGSC

SEQ ID NO: 6 - protein, 104 aa, Artificial Sequence
  Recombinant Ranpirnase variant
QDWLTFQKKHITNTRDVDCDNIMSTNLFHCKDKNTFIYSRPEPVKAICKGIIASKNVLTTSEFYLSDCNVTCRPCKYKLKKSTNKFCVTCENQAPVHFVGVGSC

SEQ ID NO: 7 - protein, 104 aa, Artificial Sequence
  Recombinant Ranpirnase variant
QDWLTFQKKHITNTRDVDCDNILSTNLFHCKDKNTFIYSRPEPVKAICKGIIASKNVLTTSEFYLSDCNVTSRPCKYKLKKSTNKFAVTCENQAPVHFVGVGSA

SEQ ID NO: 8 - protein, 104 aa, Artificial Sequence
  Recombinant Ranpirnase variant
QDWLTFQKKHITNTRDVDCDNIMSTNLFHCKDKNTFIYSRPEPVKAICKGIIASKNVLTTSEFYLSDCNVTSRPCKYKLKKSTNKFAVTCENQAPVHFVGVGSA

SEQ ID NO: 9 - protein, 104 aa, Artificial Sequence
  Recombinant Ranpirnase variant
QDWLTFQKKHITNTRDVDCDNILSTNLFHCKDKNTFIYSRPEPVKAICKGIIASKNVLTTSEFYLSDCNVTSRPCKYKLKKSTNKFCVNCANQAPVHFVGVGSC

SEQ ID NO: 10 - protein, 104 aa, Artificial Sequence
  Recombinant Ranpirnase variant
QDWLTFQKKHITNTRDVDCDNIMSTNLFHCKDKNTFIYSRPEPVKAICKGIIASKNVLTTSEFYLSDCNVTSRPCKYKLKKSTNKFCVNCANQAPVHFVGVGSC

SEQ ID NO: 11 - protein, 104 aa, Artificial Sequence
  Recombinant Ranpirnase variant
QDWLTFQKKHITNTRDVDCDNILSTNLFHAKDKNTFIYSRPEPVKAICKGIIASKNVLTTSEFYLSDCNVTSRPAKYKLKKSTNKFCVTCENQAPVHFVGVGSC

SEQ ID NO: 12 - protein, 104 aa, Artificial Sequence
  Recombinant Ranpirnase variant
QDWLTFQKKHITNTRDVDCDNIMSTNLFHAKDKNTFIYSRPEPVKAICKGIIASKNVLTTSEFYLSDCNVTSRPAKYKLKKSTNKFCVTCENQAPVHFVGVGSC

SEQ ID NO: 13 - protein, 102 aa, Artificial Sequence
  Recombinant Ranpirnase variant
QDWLTFQKKHITNTRDVDCDNILSTNLFHCKDKNTFIYSRPEPVKAICKGIIASKNVLTTSEFYLSDCNVTSRPCKYKLKKSTNKFSVTCENQAPVHFVGVG

SEQ ID NO: 14 - protein, 102 aa, Artificial Sequence
  Recombinant Ranpirnase variant
QDWLTFQKKHITNTRDVDCDNIMSTNLFHCKDKNTFIYSRPEPVKAICKGIIASKNVLTTSEFYLSDCNVTSRPCKYKLKKSTNKFSVTCENQAPVHFVGVG

SEQ ID NO: 15 - protein, 117 aa, Rana dybowskii
LFAVVLSLTHKFLCQDWKTFQDKHLTKTRDVDCDNVLSKPLFNCKDRNTFIFSRPEPVKALCKGVKDKNVLSRSEFYLSDCNVTTRHCKYKLKKKINTICITCRGEAPVHFVGVGSC

SEQ ID NO: 16 - protein, 104 aa, Rana dybowskii
QDWKTFQKKHLTKARDIKCDNIMSKTLFNCKDTNTFIFSLPGPVKALCRGIKVSKNVLSRSEFDLSECNVKSKPCKYKLKKKSDGICITCRDEAPVHFVGVGSC

SEQ ID NO: 17 - protein, 104 aa, Rana dybowskii
QDWKTFQKKHLTNTRNIKCDKIMPINLFHCKYRNTFIYSRPRGVENLCRGKINATNVSSSSKFALFECIEKSRPCKYKLKKQTNVICITCEHKVPVHFVGVGSC

SEQ ID NO: 18 - protein, 129 aa, Rana catesbeiana
MFPKFSFLLIFAIVLSLTHKSLCQDWATFKKKHLTDTWDVDCDNLMPTSLFDCKDKNTFIYSLPGPVKALCRGVIFSADVLSNSEFYLAECNVKPRKPCKYKLKKSSNRICIRCEHELPVHFAGVGICP

SEQ ID NO: 19 - protein, 106 aa, Rana catesbeiana
QDWATFKKKHLTDTWDVDCDNLMPTSLFDCKDKNTFIYSLPGPVKALCRGVIFSADVLSNSEFYLAECNVKPRKPCKYKLKKSSNRICIRCEHELPVHFAGVGICP

SEQ ID NO: 20 - protein, 128 aa, Rana catesbeiana
MFPKFSFLLIFAVVLSLTHKSLCQNWETFQKKHLTNILDINCDVEMAKALFNCKEMNTFIYALPGRVQALCKNIKDNTEVLSTDTFYLPECNRIKLPCHYKLKKPLEKICITCVNELPIHFAGVGSCP

SEQ ID NO: 21 - protein, 105 aa, Rana catesbeiana
QNWETFQKKHLTNILDINCDVEMAKALFNCKEMNTFIYALPGRVQALCKNIKDNTEVLSTDTFYLPECNRIKLPCHYKLKKPLEKICITCVNELPIHFAGVGSCP

SEQ ID NO: 22 - protein, 128 aa, Rana catesbeiana
MFPKFSFLLIFAVVLSLTHKSLCQNWETFQKKHLTNILDINCDVEMAKALFNCKEMNTFIYALPGRVQALCKNIRDNTDVLSRDAFLLPQCDRIKLPCHYKLSSSTNTICITCVNQLPIHFAGVGSCP

SEQ ID NO: 23 - protein, 105 aa, Rana catesbeiana
QNWETFQKKHLTNILDINCDVEMAKALFNCKEMNTFIYALPGRVQALCKNIRDNTDVLSRDAFLLPQCDRIKLPCHYKLSSSTNTICITCVNQLPIHFAGVGSCP

SEQ ID NO: 24 - protein, 131 aa, Rana catesbeiana
MFPKFSFLLIFAVVLSLTHKSLCQDWETFQKKHLTDTVDVNCDVEMQKALFNCKQTNTFIFARPPRVQALCKNIKDNTDVLSRDEFYLPECNRTKLPCHYKLKKPLNTICLTCRKELPVHFAGVGKCPEKV

SEQ ID NO: 25 - protein, 108 aa, Rana catesbeiana
QDWETFQKKHLTDTVDVNCDVEMQKALFNCKQTNTFIFARPPRVQALCKNIKDNTDVLSRDEFYLPECNRTKLPCHYKLKKPLNTICLTCRKELPVHFAGVGKCPEKV

SEQ ID NO: 26 - protein, 105 aa, Rana catesbeiana
QNWETFQKKHLTDTRDVKCDAEMKKALFDCKQKNTFIYARPGRVQALCKNIIVSKNVLSTDEFYLSDCNRIKLPCHYKLKKSSNTICITCENKLPVHFVAVEECP

SEQ ID NO: 27 - protein, 114 aa, Rana pipens
KPKEDREWEKFKTKHITSQSVADFNCNRTMNDPAYTPDGQCKPINTFIHSTTGPVKEICRRATGRVNKSSTQQFTLTTCKNPIRCKYSQSNTTNFICITCRDNYPVHFVKTGKC

SEQ ID NO: 28 - protein, 115 aa, Artificial Sequence
  Recombinant Amphinase-2 variant
MKPKEDREWEKFKTKHITSQSVADFNCNRTMNDPAYTPDGQCKPINTFIHSTTGPVKEICRRATGRVNKSSTQQFTLTTCKNPIRCKYSQSNTTNFICITCRDNYPVHFVKTGKC

SEQ ID NO: 29 - protein, 116 aa, Artificial Sequence
  Recombinant Amphinase-2 variant
MAKPKEDREWEKFKTKHITSQSVADFNCNRTMNDPAYTPDGQCKPINTFIHSTTGPVKEICRRATGRVNKSSTQQFTLTTCKNPIRCKYSQSNTTNFICITCRDNYPVHFVKTGKC

SEQ ID NO: 30 - protein, 121 aa, Artificial Sequence
  Recombinant Amphinase-2 variant
AAQPAMAKPKEDREWEKFKTKHITSQSVADFNCNRTMNDPAYTPDGQCKPINTFIHSTTGPVKEICRRATGRVNKSSTQQFTLTTCKNPIRCKYSQSNTTNFICITCRDNYPVHFVKTGKC

SEQ ID NO: 31 - protein, 136 aa, Artificial Sequence
  Recombinant Amphinase-2 variant
MKYLLPTAAAGLLLLAAQPAMAKPKEDREWEKFKTKHITSQSVADFNCNRTMNDPAYTPDGQCKPINTFIHSTTGPVKEICRRATGRVNKSSTQQFTLTTCKNPIRCKYSQSNTTNFICITCRDNYPVHFVKTGKC

SEQ ID NO: 32 - protein, 114 aa, Artificial Sequence
  Recombinant Amphinase-2 variant
KPKEDREWEKFKTKHITSQSVADFNCNRTMNDPAYTPDGQCKPINTFIHSTTGPVKEICRRATGRVNKSSCQQFTLTTCKNPIRCKYSQSNTTNFICITCRDNYPVHFVKTGKC

SEQ ID NO: 33 - protein, 115 aa, Artificial Sequence
  Recombinant Amphinase-2 variant
MKPKEDREWEKFKTKHITSQSVADFNCNRTMNDPAYTPDGQCKPINTFIHSTTGPVKEICRRATGRVNKSSCQQFTLTTCKNPIRCKYSQSNTTNFICITCRDNYPVHFVKTGKC

SEQ ID NO: 34 - protein, 114 aa, Artificial Sequence
  Recombinant Amphinase-2 variant
KPKEDREWEKFKTKHITSQSVADFNCNRTMNDPAYTPDGQCKPVNTFIHSTTGPVKEICRRATGRVNKSSTQQFTLTTCKNPIRCKYSQSNTTNFICITCRDNYPVHFVKTGKC

SEQ ID NO: 35 - protein, 115 aa, Artificial Sequence
  Recombinant Amphinase-2 variant
MKPKEDREWEKFKTKHITSQSVADFNCNRTMNDPAYTPDGQCKPVNTFIHSTTGPVKEICRRATGRVNKSSTQQFTLTTCKNPIRCKYSQSNTTNFICITCRDNYPVHFVKTGKC

SEQ ID NO: 36 - protein, 114 aa, Artificial Sequence
  Recombinant Amphinase-2 variant
KPKEDKEWEKFKVKHITSQSVADFNCTSTMNNPDFTPDGQCKPINTFIHSNTGPVKEICRRASGRVNKSSTQQFPLTTCKNPKRCKYSQSNETNYICITCRDNYPVHFVKIGKC

SEQ ID NO: 37 - protein, 115 aa, Artificial Sequence
  Recombinant Amphinase-2 variant
MKPKEDKEWEKFKVKHITSQSVADFNCTSTMNNPDFTPDGQCKPINTFIHSNTGPVKEICRRASGRVNKSSTQQFPLTTCKNPKRCKYSQSNETNYICITCRDNYPVHFVKIGKC

SEQ ID NO: 38 - protein, 114 aa, Artificial Sequence
  Recombinant Amphinase-2 variant
KPKEDKEWVKFKAKHITSQSVADFNCNKTMNDPDFTPDGQCKPVNTFIHSNTGPVKDICRRASGRVNKSSTQQFPLTTCNKPIRCKYSQSNTTNFICITCRDNYPVHFVKIGKC

SEQ ID NO: 39 - protein, 115 aa, Artificial Sequence
  Recombinant Amphinase-2 variant
MKPKEDKEWVKFKAKHITSQSVADFNCNKTMNDPDFTPDGQCKPVNTFIHSNTGPVKDICRRASGRVNKSSTQQFPLTTCNKPIRCKYSQSNTTNFICITCRDNYPVHFVKIGKC

(19) United States
(12) Patent Application Publication
(10) Pub. No.: US 2023/0218726 A1
(43) Pub. Date: Jul. 13, 2023

(54) TREATMENT OF VIRAL CONJUNCTIVITIS

(71) Applicant: Okogen, Inc., Encinitas, CA (US)

(72) Inventors: Brian M. Strem, Encinitas, CA (US); Eric J. Daniels, Encinitas, CA (US)

(21) Appl. No.: 17/996,577

(22) PCT Filed: Apr. 22, 2021
(86) PCT No.: PCT/US2021/028693

§ 371 Date: Oct. 19, 2022

Related U.S. Application Data
(60) Provisional application No. 63/014,380, filed on Apr. 23, 2020.

(51) Int. Cl. A61K 38/46; A61P 27/02; A61P 31/12; A61K 31/4174

(52) U.S. Cl. CPC A61K 38/465; A61P 27/02; A61P 31/12; A61K 31/4174

(57) ABSTRACT

Provided herein are compositions and methods for treating, reducing, preventing, inhibiting, mitigating, ameliorating, or slowing ocular replication or infections, such as viral conjunctivitis. Certain embodiments of the present disclosure relate to product combinations that include one or more ribonuclease, such as ranpirnase, or a variant, derivative, analogue, fragment, or pharmaceutically acceptable salt thereof, and one or more additional therapeutic agent, such as a vasoconstrictor, an antibiotic, an immunomodulatory compound, or a steroid.

### BACKGROUND

### Field

The present disclosure relates to product combinations and methods of using the same for treating, preventing, inhibiting, mitigating, ameliorating, or slowing viral ocular replication or infections. Such ocular infections include but are not limited to virus infections from Adenoviridae or Herpesviridae families of viruses.

### Background

Conjunctivitis, commonly referred to as pink eye, is an inflammation of the eye causing swelling and irritation. It affects the conjunctiva, the thin transparent membrane that covers the sclera of the eyeball and lines the inner surface of the eyelid. Conjunctivitis is most often caused by a viral or by a bacterial infection, although allergies, chemical irritants, and underlying diseases can also play a role. Symptoms of conjunctivitis include, without limitation, redness in the sclera and/or inner eyelid, ocular itching (itchy eyes), foreign body sensation (gritty or scratchy eyes), burning eyes, blurred vision, increased sensitivity to light or photophobia, swollen inner eyelids, increased tear production, watery discharge, mucopurulent discharge that can crusts over eyelashes while sleeping. Both viral and bacterial conjunctivitis are highly contagious.

While bacterial conjunctivitis typically caused by pyrogenic bacteria such as staphylococcus or streptococcus can be treated using antibiotics in the form of eye drops, pills or an ointment, there is currently no treatment for viral conjunctivitis. Viral conjunctivitis is primarily caused by viruses in the Adenoviridae family, constituting up to 90% of the viral conjunctivitis cases annually. Although adenoviral conjunctivitis is often a self-limited disease, sub-groups of patients with adenoviral conjunctivitis often have serious and sight-threatening long-term sequelae because the disease can cause corneal scarring. Herpetic viral infections, the second most common form of viral conjunctivitis, are typically more severe in nature with longer duration of contagion and associated with clinical signs and symptoms of the infection.

Thus, there is a need to develop pharmaceutical compositions and treatments for viral conjunctivitis.

### SUMMARY

Described herein are compositions, including product combinations, for use in treating, preventing, inhibiting, mitigating, ameliorating, or slowing viral ocular replication or infections, and methods of using the same for the treatment, prevention, inhibition, mitigation, amelioration, or slowing of viral ocular replication or infections. These combinations are focused to treat multiple infections, such as, for example, bacterial and viral conjunctivitis, or to enhance the therapeutic value within viral conjunctivitis, such as addressing both contagion and associated clinical signs and symptoms.

Some embodiments provided herein relate to product combinations that inhibit or slow an ocular infection. In some embodiments, the product combinations include a therapeutically effective amount of one or more ribonuclease (RNase), and a therapeutically effective amount of one or more additional therapeutic agent. In some embodiments, the additional therapeutic agent is a vasoconstrictor, an antibiotic, an immunomodulatory compound, a steroid, or a combination thereof.

In some embodiments, the one or more RNase is ranpirnase, an analogue, variant, derivative, or fragment thereof. In some embodiments, the one or more ranpirnase, analogue, variant, derivative, or fragment thereof is present in an amount of about 0.001% to about 1% w/v.

In some embodiments, the one or more additional therapeutic agent is naphazoline, tetrahydrozoline, phenylephrine, oxymetazoline, brimonidine, apraclonidine, ephedrine, azithromycin, erythromycin, gentamicin, neomycin, tobramycin, besifloxacin, ciprofloxacin, gatifloxacin, levofloxacin, moxifloxacin, ofloxacin, bacitracin, chloramphenicol, gramicidin, natamycin, polymyxin B, sulfacetamide, tetracycline, trimethoprim, vancomycin, dexamethasone, difluprednate, fluorometholone, loteprednol, prednisolone, rimexolone, cyclosporine A, an NLRP3 inhibitor, diclofenac, ketorolac, bromfenac, nepafenac, flurbiprofen, lifitegrast, or a pharmaceutically acceptable salt, analogue, or derivative thereof. In some embodiments, the NLRP3 inhibitor is Ac-YVAD-cmk, 2-APB, arglabin, BAPTA, BAY 11-7082, β-hydroxybutyrate (BHB), C172, CY-09, flufenamic acid, glybenclamide, INF39, isoliquiritigenin, MCC950, mefenamic acid, 3,4-methylenedioxy-β-nitrostyrene (MNS), OLT1177, oridonin, parthenolide, resveratrol, sulforaphane, tranilast, VX-765, or Z-VAD-FMK. In some embodiments, the additional therapeutic agent is present at a concentration of 0.001% to 5% w/v.

In some embodiments, the one or more RNase includes ranpirnase at a concentration of about 0.001% to about 1% w/v and one or more additional therapeutic agent includes naphazoline, oxymetazoline, or brimonidine at a concentration of 0.001% to 0.1% w/v.

In some embodiments, the product combination includes ranpirnase present in an amount of about 0.03% w/v and oxymetazoline present in an amount of about 0.01% to about 0.025% w/v. In some embodiments, the product combination includes ranpirnase present in an amount of about 0.03% w/v and brimonidine present in an amount of about 0.01% to about 0.025% w/v.

In some embodiments, the ocular infection is viral conjunctivitis. In some embodiments, the viral conjunctivitis is epidemic keratoconjunctivitis, pharyngoconjunctival fever, nonspecific sporadic follicular conjunctivitis, or chronic papillary conjunctivitis. In some embodiments, the viral conjunctivitis is caused by a virus infection from the Adenoviridae or Herpesviridae family, such as, for example, Human adenovirus B, a Human adenovirus D, a Human adenovirus E, herpes simplex virus (HSV), varicella zoster virus (VZV), Epstein-Barr virus (EBV), human cytomegalovirus (CMV), or herpes zoster virus (HZV). In some embodiments, the Human adenovirus B is a Human adenovirus B serotype 3, a Human adenovirus B serotype 7, a Human adenovirus B serotype 11, or any combination thereof. In some embodiments, the Human adenovirus D is a Human adenovirus D serotype 8, a Human adenovirus D serotype 13, a Human adenovirus D serotype 19, a Human adenovirus D serotype 37, or any combination thereof. In some embodiments, the Human adenovirus E is a Human adenovirus E serotype 4.

In some embodiments, the product combinations further include one or more pharmaceutically acceptable carriers and optionally one or more pharmaceutically acceptable components.

In some embodiments, the one or more RNase and the one or more additional therapeutic agent are formulated in a single formulation or a single dosage. In some embodiments, the one or more RNase is prepared in a first composition and the one or more additional therapeutic agent is prepare prepared in a second composition. In some embodiments, the first composition is separate from the second composition. In some embodiments, the product combination is formulated as an ophthalmic formulation for use in an ophthalmic route of administration. In some embodiments, the product combination is formulated for administration by ocular instillation, ocular irrigation, intraocular injection, intracorneal injection, intravitreal injection, or subconjunctival injection. In some embodiments, the product combination is a controlled release delivery platform. In some embodiments, the controlled release delivery platform is an extended-release formulation or a sustained release formulation. In some embodiments, the product combination is an ocular implant, an ophthalmic implant, a punctal plug, an intraocular implant, an intracorneal implant, or a subconjunctival implant. In some embodiments, the product combination is administered two times a day. In some embodiments, the product combination is administered four times a day. In some embodiments, the product combination is administered eight times a day. In some embodiments, the product combination comprises ranpirnase in an amount of about 25 mM and oxymetazoline in an amount of 0.01% to 0.025% w/v.

Some embodiments provided herein relate to methods of reducing or inhibiting an ocular infection in a subject. In some embodiments, the method includes selecting a subject in need of a product combination that reduces or inhibits an ocular infection and administering to the subject a product combination including a therapeutically effective amount of one or more ribonuclease (RNase), and a therapeutically effective amount of one or more additional therapeutic agent. In some embodiments, the product combination is any product combination as described herein. In some embodiments, the additional therapeutic agent is a vasoconstrictor, an antibiotic, an immunomodulatory compound, a steroid, or a combination thereof. In some embodiments, the one or more RNase is ranpirnase, an analogue, variant, derivative, or fragment thereof. In some embodiments, the one or more additional therapeutic agent is naphazoline, tetrahydrozoline, phenylephrine, oxymetazoline, brimonidine, apraclonidine, ephedrine, azithromycin, erythromycin, gentamicin, neomycin, tobramycin, besifloxacin, ciprofloxacin, gatifloxacin, levofloxacin, moxifloxacin, ofloxacin, bacitracin, chloramphenicol, gramicidin, natamycin, polymyxin B, sulfacetamide, tetracycline, trimethoprim, vancomycin, dexamethasone, difluprednate, fluorometholone, loteprednol, prednisolone, rimexolone, cyclosporine A, an NLRP3 inhibitor, diclofenac, ketorolac, bromfenac, nepafenac, flurbiprofen, lifitegrast, or a pharmaceutically acceptable salt, analogue, or derivative thereof. In some embodiments, the NLRP3 inhibitor is Ac-YVAD-cmk, 2-APB, arglabin, BAPTA, BAY 11-7082, β-hydroxybutyrate (BHB), C172, CY-09, flufenamic acid, glybenclamide, INF39, isoliquiritigenin, MCC950, mefenamic acid, 3,4-methylenedioxy-β-nitrostyrene (MNS), OLT1177, oridonin, parthenolide, resveratrol, sulforaphane, tranilast, VX-765, or Z-VAD-FMK.

In some embodiments, the methods include administration of a product combination that includes ranpirnase present in an amount of about 0.03% w/v and oxymetazoline present in an amount of about 0.01% to about 0.025% w/v. In some embodiments, the methods include administration of a product combination that includes ranpirnase present in an amount of about 0.03% w/v and brimonidine present in an amount of about 0.01% to about 0.025% w/v.

In some embodiments, the method inhibits or delays the ocular infection or prevents spread of the ocular infection. In some embodiments, the ocular infection is a viral conjunctivitis. In some embodiments, the viral conjunctivitis is epidemic keratoconjunctivitis, pharyngoconjunctival fever, nonspecific sporadic follicular conjunctivitis, chronic papillary conjunctivitis, or herpetic conjunctivitis. In some embodiments, the product combination is administered to the subject ophthalmically. In some embodiments, the one or more RNase is prepared in a first composition and the one or more additional therapeutic agent is prepared in a second composition. In some embodiments, the first composition is administered prior to, concomitantly with, or subsequent to administration of the second composition. In some embodiments, the administering is two times a day. In some embodiments, the administering is four times a day. In some embodiments, the administering is eight times a day.

Some embodiments provided herein relate to uses of a product combination in the manufacture of a medicament for the treatment of an ocular infection. In some embodiments, the product combination includes a therapeutically effective amount of one or more ribonuclease (RNase), and a therapeutically effective amount of one or more additional therapeutic agent. In some embodiments, the product combination is any product combination as described herein. In some embodiments, the additional therapeutic agent is a vasoconstrictor, an antibiotic, an immunomodulatory compound, a steroid, or a combination thereof. In some embodiments, the one or more RNase is ranpirnase, an analogue, variant, derivative, or fragment thereof. In some embodiments, the one or more additional therapeutic agent is naphazoline, tetrahydrozoline, phenylephrine, oxymetazoline, brimonidine, apraclonidine, ephedrine, azithromycin, erythromycin, gentamicin, neomycin, tobramycin, besifloxacin, ciprofloxacin, gatifloxacin, levofloxacin, moxifloxacin, ofloxacin, bacitracin, chloramphenicol, gramicidin, natamycin, polymyxin B, sulfacetamide, tetracycline, trimethoprim, vancomycin, dexamethasone, difluprednate, fluorometholone, loteprednol, prednisolone, rimexolone, cyclosporine A, an NLRP3 inhibitor, diclofenac, ketorolac, bromfenac, nepafenac, flurbiprofen, lifitegrast, or a pharmaceutically acceptable salt, analogue, or derivative thereof. In some embodiments, the NLRP3 inhibitor is Ac-YVAD-cmk, 2-APB, arglabin, BAPTA, BAY 11-7082, β-hydroxybutyrate (BHB), C172, CY-09, flufenamic acid, glybenclamide, INF39, isoliquiritigenin, MCC950, mefenamic acid, 3,4-methylenedioxy-β-nitrostyrene (MNS), OLT1177, oridonin, parthenolide, resveratrol, sulforaphane, tranilast, VX-765, or Z-VAD-FMK. In some embodiments, the medicament inhibits or delays the ocular infection. In some embodiments, the ocular infection is a viral conjunctivitis. In some embodiments, the viral conjunctivitis is epidemic keratoconjunctivitis, pharyngoconjunctival fever, nonspecific sporadic follicular conjunctivitis, or chronic papillary conjunctivitis. In some embodiments, the medicament is formulated for ophthalmic administration. In some embodiments, the product combination comprises ranpirnase present in an amount of about 0.03% w/v and oxymetazoline present in an amount of about 0.01% to about 0.025% w/v. In some embodiments, the product combination comprises ranpirnase present in an amount of about 0.03% w/v and brimonidine present in an amount of about 0.01% to about 0.025% w/v.

### DETAILED DESCRIPTION

Embodiments provided herein relate to product combinations for use in treating, preventing, inhibiting, mitigating, ameliorating, or slowing viral ocular replication or infections. In some embodiments, the product combination includes one or more ribonuclease in combination with one or more vasoconstrictor, antibiotic, immunomodulatory compound, including steroids or non-steroidal anti-inflammatory drugs (NSAIDs), or any combination thereof. Some embodiments provided herein relate to methods of using the product combination for the treatment, prevention, inhibition, mitigation, amelioration, or slowing of viral ocular replication or infections.

It will be readily understood that the aspects of the present disclosure, as generally described herein, can be arranged, substituted, combined, separated, and designed in a wide variety of different configurations, all of which are explicitly contemplated herein.

Unless defined otherwise, technical and scientific terms used herein have the same meaning as commonly understood by one of ordinary skill in the art to which the present disclosure belongs. All patents, applications, published applications and other publications referenced herein are expressly incorporated by reference in their entireties unless stated otherwise. For purposes of the present disclosure, the following terms are defined below.

By “about” is meant a quantity, level, value, number, frequency, percentage, dimension, size, amount, weight, or length that varies by as much as 30, 25, 20, 15, 10, 9, 8, 7, 6, 5, 4, 3, 2 or 1% to a reference quantity, level, value, number, frequency, percentage, dimension, size, amount, weight, or length. When a value is preceded by the term about, the component is not intended to be limited strictly to that value, but it is intended to include amounts that vary from the value.

Throughout this specification, unless the context requires otherwise, the words “comprise,” “comprises,” and “comprising” may be understood to imply the inclusion of a stated step or element or group of steps or elements but not the exclusion of any other step or element or group of steps or elements.

### I. Ocular Infections

Embodiments provided herein relate to compositions and methods for treating, preventing, inhibiting, reducing, mitigating, ameliorating, or slowing viral replication or infections of the eye. In particular, the compositions and methods relate to treating, preventing, inhibiting, reducing, mitigating, ameliorating, or slowing viral replication or infections of the eye. In some embodiments, the ocular infection is a viral infection caused by a virus in the Adenoviridae family, including for example, but not limited to, adenovirus types 3, 4, 7, 8, 19, 29, 37, or 54. In some embodiments, the ocular infection is a viral infection caused by a virus in the Herpesviridae family, including for example, herpes simplex virus 1 or 2 (HSV-1 or HSV-2), Epstein-Barr virus (EBV), human cytomegalovirus (CMV), herpes zoster virus (HZV), or varicella zoster virus (VZV).

Adenoviruses (members of the family Adenoviridae) are medium-sized (90 to 100 nm), non-enveloped viruses with an icosahedral nucleocapsid containing a double-stranded DNA genome. Adenoviruses have a broad range of vertebrate hosts. About 60 distinct adenoviral serotypes have been found to cause a wide range of illnesses in humans, from mild respiratory infections in young children (known as the common cold) to life-threatening multi-organ disease in people with a weakened immune system.

Viral infections of the eye can have significant consequences. With respect to viral conjunctivitis adenovirus serotypes 3, 4, 7, 8, 11, 13, 19, 37, and 54 appear to be the primary causative agents, although other adenovirus serotypes may also cause viral conjunctivitis. Adenovirus serotypes 3, 7 and 11 are classified as Human adenovirus B; serotypes 8, 13, 19 and 37 are classified as Human adenovirus D; and serotype 4 is classified as Human adenovirus E. Because of low natural immunity against adenovirus in the general population, every individual is considered to be susceptible to infection. In addition, conjunctival viral infections initiate a strong innate and adaptive immune response. The ability to modulate this immune response may aid in reducing many of the clinical signs and symptoms associated with viral conjunctivitis.

Clinically, these adenoviruses can cause multiple distinct syndromes, with epidemic keratoconjunctivitis (EKC—primarily serotypes 8, 19, 37) and pharyngoconjunctival fever (PCF—primarily serotypes 3, 4, 7) being the most common. EKC is one of the most common syndromes of acute conjunctivitis, with characteristic clinical features such as sudden onset of acute follicular conjunctivitis, with watery discharge, hyperemia (redness), chemosis, and ipsilateral preauricular lymphadenopathy. Corneal involvement can occur in the form of diffuse, fine, and/or superficial keratitis, epithelial defects, and even subepithelial infiltrates and opacities. In 20-50% of cases, corneal opacities can persist for months. These sequelae can significantly decrease visual acuity and cause glare symptoms. Treatment is mostly intended to control symptoms through the use of cold compresses and artificial tears. Antivirals (such as cidofovir) and cyclosporine eye drops were tested clinically but no definitive benefit was observed. In very specific cases with severe membranous involvement, mild topical corticosteroids can be used to control inflammation.

Different viruses of the Herpesviridae family can infect various tissues of the eye. Type 1 and type 2 herpes simplex viruses (HSV-1 and HSV-2) infect the front of the eye and cause conjunctivitis and keratitis. CMV is known to infect the back of the eye, causing retinitis. HZV can cause chronic, severe eye disease when affecting the trigeminal area. Herpes zoster ophthalmicus, a severe form of acute herpes zoster, results from the reactivation of VZV in the trigeminal (fifth cranial) nerve.

The product combinations provided herein, comprising one or more RNase, such as one or more ranpirnase, amphinase, variant, analogue, derivative, or fragment thereof and one or more additional therapeutic agent, such as one or more vasoconstrictor, one or more antibiotic, one or more immunomodulatory compound, or one or more steroid, or a combination thereof may be used for the treatment of a viral ocular replication or infection as described herein, such as a viral conjunctivitis. A viral conjunctivitis disclosed herein includes an epidemic keratoconjunctivitis, a pharyngoconjunctival fever, a nonspecific sporadic follicular conjunctivitis, or a chronic papillary conjunctivitis. A viral conjunctivitis disclosed herein may be caused by any virus replication or virus infection disclosed herein or known by those of skill in the art, including, for example, a virus infection from the Adenoviridae or Herpesviridae family, such as, for example, Human adenovirus B, a Human adenovirus D, a Human adenovirus E, HSV, VZV, EBV, HZV, or CMV.

### II. Compositions

Some embodiments provided herein relate to product combinations for treating, preventing, inhibiting, reducing, mitigating, ameliorating, or slowing ocular replication or infections. In some embodiments, the ocular infection is a viral infection, including, for example, a viral infection from a virus of the Adenoviridae or Herpesviridae family. In some embodiments, the product combination includes one or more ribonuclease enzyme and one or more vasoconstrictor, antibiotic, immunomodulatory compound, or steroid, or a combination thereof.

As used herein, the terms “treating,” “treatment,” “therapeutic,” or “therapy” have their ordinary meaning as understood in light of the specification, and do not necessarily mean total cure or abolition of the disease or condition.

As used herein, the term “inhibit” has its ordinary meaning as understood in light of the specification and refers to the delay or prevention of a viral ocular replication or infection, such as a viral infection caused by a virus of the Adenoviridae or Herpesviridae family. As used herein, the term “delay” has its ordinary meaning as understood in light of the specification, and refers to a slowing, postponement, or deferment of an event, such as the delay of an ocular replication or infection, such as a viral infection caused by a virus of the Adenoviridae or Herpesviridae family, to a time that is later than would otherwise be expected. The delay can be a delay of 10%, 20%, 30%, 40%, 50%, 60%, 70%, 80%, 90%, 100%, or an amount within a range defined by any two of the aforementioned values. The terms inhibit and delay are not to be construed as necessarily indicating a 100% inhibition or delay. A partial inhibition or delay may be realized.

The term “therapeutically effective amount” has its ordinary meaning as understood in light of the specification and is used to indicate an amount of an active compound, or pharmaceutical agent, that elicits the biological or medicinal response indicated. For example, a therapeutically effective amount of compound can be the amount needed to prevent, alleviate, mitigate, or ameliorate a viral ocular replication or infection. This response may occur in a tissue, system, animal, or human and includes alleviation of the signs or symptoms of the disease being treated. Determination of a therapeutically effective amount is within the capability of those skilled in the art, in view of the disclosure provided herein. The therapeutically effective amount of the compounds disclosed herein required as a dose may depend on the route of administration, the type of animal, including human, being treated, and the physical characteristics of the specific animal under consideration. The dose can be tailored to achieve a desired effect, but may depend on such factors as weight, diet, concurrent medication, and other factors that those skilled in the medical arts will recognize.

As used herein, the term “derivative” has its ordinary meaning as understood in light of the specification and refers to a chemically modified compound wherein the modification is considered routine by the ordinary skilled chemist, such as an ester or an amide of an acid or protecting groups such as a benzyl group for an alcohol or thiol, or a tert-butoxycarbonyl group for an amine.

As used herein, the term “analogue” has its ordinary meaning as understood in light of the specification and refers to a compound, which includes a chemically modified form of a specific compound or class thereof and which maintains the pharmaceutical and/or pharmacological activities characteristic of said compound or class.

As used herein, a “ribonuclease enzyme,” “ribonuclease,” or “RNase” has its ordinary meaning as understood in light of the specification and is used to describe a nuclease that catalyzes degradation of RNA into smaller components. In some embodiments, the RNase is a ranpirnase or an amphinase, a variant form thereof, a recombinant form thereof, or a fraction thereof.

Ranpirnase is an amphibian ribonuclease originally isolated from oocytes and/or early embryos of the Rana pipiens (the Northern Leopard frog). Originally called P-30 Protein or P-30, ranpirnase is a member of the pancreatic ribonuclease (RNase A) protein superfamily. Initially expressed as a precursor polypeptide, ranpirnase is processed to remove both the precursor peptide portion and the start methionine to produce a basic, lysine-rich, enzyme having a molecular weight of about 12 kD. The N-terminal pyroglutamyl residue is an integral part of ranpirnase active site and significantly contributing to the catalytic and biological activities of ranpirnase as well as to its unusually high conformational stability. Another structural feature of ranpirnase is the C-terminal disulfide bond (87-104) that stabilizes the protein compact structure. This, in turn, makes ranpirnase highly resistant to endogenous proteases. Another feature of ranpirnase that makes it resistant to endogenous proteases is the low intracellular binding affinity observed for specific RNase inhibitors, allowing ranpirnase to remain active inside the cell while the majority of mammalian RNases are inhibited.

Ranpirnase primarily targets rapidly replicating and/or growing cells by binding to cell surface receptors and internalizing into the cytoplasm via AP-2/clathrin-mediated endocytosis. The enzyme is then shuttled to the endoplasmic reticulum where it degrades RNA substrates with a sequence preference for uracil and guanine nucleotides. For example, cleavage site mapping using natural Transfer RNA (tRNA) substrates in vitro revealed predominant cleavage sites at UG and GG residues as well as cleavage at CG sites. Transfer RNA appears to be preferentially targeted as a substrate by ranpirnase, which leaves messenger RNA (mRNA) and ribosomal RNA (rRNA) undamaged. The degradation of tRNA by ranpirnase results in the inhibition of protein synthesis.

However, the biological effects of ranpirnase cannot be explained solely by a decline in protein synthesis suggesting that additional or alternative RNA molecules may be targeted by ranpirnase. One alternative mechanism has been attributed to the RNA interference pathway and the degradation of miRNAs, siRNAs, or precursors thereof. These small RNAs, similar to tRNAs, are unprotected by proteins and may also be degraded by ranpirnase. Ranpirnase may also degrade the precursors of small RNAs and thus, generate siRNAs and affect gene expression. Recent findings revealed a new class of regulatory RNAs (30-40 nt) that may be derived from small non-coding RNAs, especially tRNA, suggesting that ranpirnase could generate siRNAs directly from its intracellular tRNA substrate.

Ranpirnase has also been shown to possess immunomodulatory mechanisms of action through interference with the nuclear factor kappa-light-chain-enhancer of activated B cells (NFκB) pathway. An in vitro study showed that ranpirnase inhibits translocation of NFκB into the nucleus. NFκB, a protein complex that controls transcription of DNA, is of a key master regulator of inflammation in response to proinflammatory stimulation. NFκB is found in almost all animal cells and regulates cellular responses to stimuli such as stress, free radicals, bacterial and/or viral antigens. Further, NFκB plays a key role in regulating the immune response to infection (κ light chains are important components of immunoglobulins). By inhibiting the translocation of NFκB into the nucleus, where it is required in order to enhance inflammation, the inflammatory process may be dampened. In addition, tumor necrosis factor alpha (TNFα) and Interleukin 1-b (IL-1b) are activated by NFκB in a positive feedback loop in which genes that are regulated by NFκB also cause the activation of NFκB. Proinflammatory cytokines (including TNFα and IL-1b) attract inflammatory cells to sites of inflammation, enzymes which generate mediators of inflammation, immune receptors, and adhesion molecules that play an important role in the initial recruitment of neutrophils and macrophages to sites of inflammation. Thus, the activation and translocation of NFκB therefore leads to a coordinated increase in the expression of many genes whose products mediate inflammatory and immune responses. This type of positive regulatory loop may amplify and perpetuate local inflammatory responses. As such, the finding that ranpirnase blocked the proinflammatory effects mediated by NFκB activity suggests that this enzyme could effectively suppress an inflammatory response. It is interesting to note that in both mice and humans with either an acute trauma (mice) or epidemic keratoconjunctivitis (patient that was diagnosed with adenoviral infection), NFκB translocated into the nucleus from the cytoplasm of conjunctival epithelial cells. This is of critical observation as a drug, such as ranpirnase, that has the ability of block such translocation, has the ability to knock down the inflammatory response to such triggers.

In some embodiments, the ranpirnase disclosed herein is a wild type ranpirnase, a recombinant ranpirnase, a ranpirnase variant, a ranpirnase fraction, or an analogue thereof. In some embodiments, the ranpirnase comprises an amino acid sequence as set forth in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, or a ranpirnase having an amino acid identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

In some embodiments, the ranpirnase has an amino acid identity in the range of about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

In some embodiments, the ranpirnase has at least 1, 2, 3, 4, 5, 6, 7, 8, 9, 10, 11, 12, 13, 14, or 15 contiguous amino acid deletions, additions, and/or substitutions relative to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26; or at most 1, 2, 3, 4, 5, 6, 7, 8, 9, 10, 11, 12, 13, 14, or 15 contiguous amino acid deletions, additions, and/or substitutions relative to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

In some embodiments, the ranpirnase has at least 1, 2, 3, 4, 5, 6, 7, 8, 9, 10, 11, 12, 13, 14, or 15 non-contiguous amino acid deletions, additions, and/or substitutions relative to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26; or at most 1, 2, 3, 4, 5, 6, 7, 8, 9, 10, 11, 12, 13, 14, or 15 non-contiguous amino acid deletions, additions, and/or substitutions relative to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

In some embodiments, a ranpirnase disclosed herein can have the N-terminus blocked with pyroglutamic acid (PCA). In some embodiments, the pyroglutamic acid N-terminus block is produced by autocyclization of glutamine (Gln). In some embodiments, a ranpirnase disclosed herein can also have the N-terminus blocked with pyrrolidone carboxylic acid. In some embodiments, a ranpirnase comprising an amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26 has its N-terminus blocked with pyroglutamic acid or pyrrolidone carboxylic acid.

Amphinase is a member of the pancreatic RNase A protein superfamily. Amphinase was also isolated from amphibians and is a more basic variant of ranpirnase. Initially expressed as a precursor polypeptide, amphinase is processed to remove both the precursor peptide portion and the start methionine to produce an active enzyme having a molecular weight of about 13 kD. Like ranpirnase, amphinase primarily targets rapidly replicating and/or growing cells by degrading RNA and at a minimum inhibit protein synthesis.

In some embodiments, the amphinase disclosed herein is a wild type amphinase, a recombinant amphinase, an amphinase variant, an amphinase fraction, or an analogue thereof. In some embodiments, the amphinase comprises an amino acid sequence as set forth in any one of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 39. In some embodiments, the amphinase has an amino acid identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to any one of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 39. In some embodiments, the amphinase has an amino acid identity in the range of about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any one of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 39.

In some embodiments, the amphinase has at least 1, 2, 3, 4, 5, 6, 7, 8, 9, 10, 11, 12, 13, 14, or 15 contiguous amino acid deletions, additions, and/or substitutions relative to any one of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 39; or at most 1, 2, 3, 4, 5, 6, 7, 8, 9, 10, 11, 12, 13, 14, or 15 contiguous amino acid deletions, additions, and/or substitutions relative to any one of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 39. In some embodiments, the amphinase has at least 1, 2, 3, 4, 5, 6, 7, 8, 9, 10, 11, 12, 13, 14, or 15 non-contiguous amino acid deletions, additions, and/or substitutions relative to any one of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 39; or at most 1, 2, 3, 4, 5, 6, 7, 8, 9, 10, 11, 12, 13, 14, or 15 non-contiguous amino acid deletions, additions, and/or substitutions relative to any one of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 39.

Other RNase compounds that may be used in the compositions and methods described herein include, for example, compounds as disclosed in U.S. Pat. Nos. 5,559,212, 5,728,805, 6,239,257, 6,175,003, 6,423,515, 7,229,824, 7,442,535, 7,442,536, 7,473,542, 7,556,953, 7,585,655, 7,763,449, 7,556,951, 7,556,952, 7,585,654, 8,518,399, 8,663,964, 8,808,690, and 9,682,130, each of which is incorporated by reference in its entirety and for the specific disclosure referenced herein.

In some embodiments, the RNase, such as ranpirnase or amphinase disclosed herein is recombinantly engineered. In some embodiments, a recombinant RNase adds functional domains without inhibiting the endogenous activity of the RNase. For instance, an Eosinophilic Cationic Protein fragment can be added to the RNase in order to provide or significantly improve bactericidal properties. Such constructs are described in Torrent, et al., “Bactericidal Activity Engineered on Human Pancreatic Ribonuclease and Onconase”, Mol. Pharm. 6(2): 531-542 (2009), which is incorporated by reference in its entirety. This could be of significant value if a single therapeutic could treat both bacterial and viral conjunctivitis, while still addressing the common underlying immune response to such stress.

In some embodiments, an RNase polypeptide includes a variant polypeptide where one amino acid is added, deleted, or substituted for another. A substitution can be assessed by a variety of factors, such as, e.g., the physic properties of the amino acid being substituted or how the original amino acid would tolerate a substitution. The selections of which amino acid can be substituted for another amino acid in a polypeptide are known to a person of ordinary skill in the art.

In some embodiments, the product combinations provided herein further comprise one or more additional therapeutic agent, such as one or more vasoconstrictor, antibiotic, immunomodulatory compound, or steroid, or a derivative, analogue, or pharmaceutically acceptable salt thereof.

As used herein, a “vasoconstrictor” has its ordinary meaning as understood in light of the specification, and refers to a compound that causes vasoconstriction, or narrowing of blood vessels, including capillaries, when administered to a subject. A vasoconstrictor may include, for example, an adrenergic receptor agonist, such as:

Naphazoline (including pharmaceutically acceptable salts thereof. Naphazoline includes Naphcon or 2-(naphthalen-1-ylmethyl)-4,5-dihydro-1H-imidazole);

Tetrahydrozoline (including pharmaceutically acceptable salts thereof. Tetrahydrozoline includes tetryzoline or (RS)-2-(1,2,3,4-tetrahydronaphthalen-1-yl)-4,5-dihydro-1H-imidazole);

Phenylephrine (including pharmaceutically acceptable salts thereof. Phenylephrine includes Mydfrin, Altafrin, AK-dilate, Neofrin, (R)-3-[-1-hydroxy-2-(methylamino)ethyl]phenol);

Oxymetazoline (including pharmaceutically acceptable salts thereof. Oxymetazoline includes Afrin, Ocuclear, Drixine, 3-(4,5-dihydro-1H-imidazol-2-ylmethyl)-2,4-dimethyl-6-tert-butyl-phenol);

Brimonidine (including pharmaceutically acceptable salts thereof. Brimonidine includes Alphagan, Mirvaso, Lumify, 5-Bromo-N-(4,5-dihydro-1H-imidazol yl) quinoxalin-6-amine);

Apraclonidine (including pharmaceutically acceptable salts thereof. Apraclonidine includes Iopidine, 2,6-Dichloro-N-(4,5-dihydro-1H-imidazol-2-yl) benzene-1,4-diamine); or

Ephedrine (including pharmaceutically acceptable salts thereof. Ephedrine includes Bronkaid, Primatene, rel-(R,S)-2-(methylamino)-1-phenylpropan-1-ol)

or derivatives, salts, or analogues thereof, or combinations thereof.

As used herein, an “antibiotic” has its ordinary meaning as understood in light of the specification and refers to a compound that is used to treat and/or prevent bacterial infection by killing bacteria, inhibiting the growth of bacteria, or reducing the viability of bacteria. An antibiotic may include, for example, a macrolide, an aminoglycoside, a fluoroquinolone, or other antibiotic, such as:

Azithromycin (including pharmaceutically acceptable salts thereof. Azithromycin includes Zithromax, Azithrocin, 2R,3S,4R,5R,8R,10R,11R,12S,13S,14R)-2-ethyl-3,4,10-trihydroxy-3,5,6,8,10,12,14-heptamethyl-15-oxo-11-{[3,4,6-trideoxy-3-(dimethylamino)-β-D-xylo-hexopyranosyl]oxy}-1-oxa-6-azacyclopentadec-13-yl 2,6-dideoxy-3C-methyl-3-O-methyl-α-L-ribo-hexopyranoside, 9-deoxy-9α-aza-9α-methyl-9α-homoerythromycin A);

Erythromycin (including pharmaceutically acceptable salts thereof. Erythromycin includes Eryc, Erythrocin, 3R,4S,5S,6R,7R,9R,11R,12R,13S,14R)-6-{[(2S,3R,4S,6R)-4-(dimethylamino)-3-hydroxy-6-methyloxan-2-yl]oxy}-14-ethyl-7,12,13-trihydroxy-4-{[(2R,4R,5S,6S)-5-hydroxy-4-methoxy-4,6-dimethyloxan-2-yl]oxy}-3,5,7,9,11,13-hexamethyl-1-oxacyclotetradecane-2,10-dione);

Gentamicin (including pharmaceutically acceptable salts thereof. Gentamicin includes Cidomycin, Septopal, Genticyn, Garamycin, 3R,4R,5R)-2-{[(1S,2S,3R,4S,6R)-4,6-diamino-3-{[(2R,3R,6S)-3-amino-6-[(1R)-1-(methylamino)ethyl]oxan-2-yl]oxy}-2-hydroxycyclohexyl]oxy}-5-methyl-4-(methylamino)oxane-3,5-diol);

Neomycin (including pharmaceutically acceptable salts thereof. Neomycin includes Neo-rx, 2RS,3S,4S,5R)-5-Amino-2-(aminomethyl)-6-((2R,3S,4R,5S) ((1R,2R,5R,6R)-3,5-diamino-2-((2R,3S,4R,5S)-3-amino-6-(aminomethyl)-4,5-dihydroxytetrahydro-2H-pyran-2-yloxy)-6-hydroxycyclohexyloxy)-4-hydroxy (hydroxymethyl)tetrahydrofuran-3-yloxy) tetrahydro-2H-pyran-3,4-diol);

Tobramycin (including pharmaceutically acceptable salts thereof. Tobramycin includes Tobrex, Tobi, 2S,3R,4S,5S,6R)-4-amino-2-{[(1S,2S,3R,4S,6R)-4,6-diamino-3-{[(2R,3R,5S,6R)-3-amino-6-(aminomethyl)-5-hydroxyoxan-2-yl]oxy}-2-hydroxycyclohexyl]oxy}-6-(hydroxymethyl)oxane-3,5-diol);

Besifloxacin (including pharmaceutically acceptable salts thereof. Besifloxacin includes Besivance, 7-[(3R)-3-Aminoazepam-1-yl]-8-chloro-1-cyclopropyl-6-fluoro-4-oxo-1,4-dihydroquinoline-3-carboxylic acid);

Ciprofloxacin (including pharmaceutically acceptable salts thereof. Ciprofloxacin includes Ciloxan, Cipro, Neofloxin, 1-cyclopropyl-6-fluoro-4-oxo-7-(piperazin-1-yl)-quinoline-3-carboxylic acid);

Gatifloxacin (including pharmaceutically acceptable salts thereof. Gatifloxacin includes Gatiflo, Tequin, Zymar, 1-Cyclopropyl-6-fluoro-8-methoxy-7-(3-methylpiperazin-1-yl)-4-oxo-quinoline-3-carboxylic acid);

Levofloxacin (including pharmaceutically acceptable salts thereof. Levofloxacin includes Levaquin, Tavanic, Iquix, (S)-9-fluoro-2,3-dihydro-3-methyl-10-(4-methylpiperazin-1-yl)-7-oxo-7H-pyrido[1,2,3-de]-1,4-benzoxazine-6-carboxylic acid);

Moxifloxacin (including pharmaceutically acceptable salts thereof. Moxifloxacin includes Avelox, Vigamox, Moxeza, 1-Cyclopropyl-7-[(1S,6S)-2,8-diazabicyclo[4.3.0]nonan-8-yl]-6-fluoro-8-methoxy-4-oxoquinoline-3-carboxylic acid);

Ofloxacin (including pharmaceutically acceptable salts thereof. Ofloxacin includes Floxin, Ocuflox, (±)-9-fluoro-2,3-dihydro-3-methyl-10-(4-methyl-1-piperazinyl)-7-oxo-7H-pyrido[1,2,3-de][1,4]benzoxazine-6-carboxylic acid, (RS)-7-fluoro-2-methyl-6-(4-methylpiperazin-1-yl)-10-oxo-4-oxa-1-azatricyclo[7.3.1.0^{5,13}]trideca-5(13),6,8,11-tetraene-11-carboxylic acid);

Bacitracin (including pharmaceutically acceptable salts thereof. Bacitracin includes Baciim, (4R)-4-[(2S)-2-({2-[(1S)-1-amino-2-methylbutyl]-4,5-dihydro-1,3-thiazol-5-yl}formamido)-4-methylpentanamido]-4-{[(1S)-1-{[(3S,6R,9S,12R,15S,18R,21S)-18-(3-aminopropyl)-12-benzyl-15-(butan-2-yl)-3-(carbamoylmethyl)-6-(carboxymethyl)-9-(1H-imidazol-5-ylmethyl)-2,5,8,11,14,17,20-heptaoxo-1,4,7,10,13,16,19-heptaazacyclopentacosan-21-yl]carbamoyl}-2-methylbutyl]carbamoyl}butanoic acid);

Chloramphenicol (including pharmaceutically acceptable salts thereof. Chloramphenicol includes Pentamycetin, Chloromycetin, 2,2-dichloro-N-[(1R,2R)-1,3-dihydroxy-1-(4-nitrophenyl)propan-2-yl]acetamide);

Gramicidin (including pharmaceutically acceptable salts thereof. Gramicidin includes Bacillus brevis gramicidin D, having a linear pentadecapeptide chain of formyl-L-X-Gly-L-Ala-D-Leu-L-Ala-D-Val-L-Val-D-Val-L-Trp-D-Leu-L-Y-D-Leu-L-Trp=D-Lei-L-Trp-ethanolamine);

Natamycin (including pharmaceutically acceptable salts thereof. Natamycin includes Natacyn, 1R,3S,5R,7R,8E,12R,14E,16E,18E,20E,22R,24S,25R,26S)-22-[(3-amino-3,6-dideoxy-D-mannopyranosyl)oxy]-1,3,26-trihydroxy-12-methyl-10-oxo-6,11,28-trioxatricyclo[22.3.1.0^{5,7}]octacosa-8,14,16,18,20-pentaene-25-carboxylic acid);

Polymyxin B (including pharmaceutically acceptable salts thereof. Polymyxin B includes N-[4-amino-1-[[1-[[4-amino-1-oxo-1-[[6,9,18-tris(2-aminoethyl)-15-benzyl-3-(1-hydroxyethyl)-12-(2-methylpropyl)-2,5,8,11,14,17,20-heptaoxo-1,4,7,10,13,16,19-heptazacyclotricos-21-yl]amino]butan-2-yl]amino]-3-hydroxy-1-oxobutan-2-yl]amino]-1-oxobutan-2-yl]-6-methyloctanamide);

Sulfacetamide (including pharmaceutically acceptable salts thereof. Sulfacetamide includes Bleph-10, N-[(4-aminophenyl)sulfonyl]acetamide);

Tetracycline (including pharmaceutically acceptable salts thereof. Tetracycline includes Sumycin, (4S,6S,12aS)-4-(dimethylamino)-1,4,4a,5,5a,6,11,12a-octahydro-3,6,10,12,12a-pentahydroxy-6-methyl-1,11-dioxonaphthacene-2-carboxamide);

Trimethoprim (including pharmaceutically acceptable salts thereof. Trimethoprim includes Proloprim, Monotrim, Triprim, 5-(3,4,5-Trimethoxybenzyl)pyrimidine-2,4-diamine);

Vancomycin (including pharmaceutically acceptable salts thereof. Vancomycin includes Vancocin, (1S,2R,18R,19R,22S,25R,28R,40S)-48-{[(2S,3R,4S,5S,6R)-3-{[(2S,4S,5S,6S)-4-amino-5-hydroxy-4,6-dimethyloxan-2-yl]oxy}-4,5-dihydroxy-6-(hydroxymethyl)oxan-2-yl]oxy}-22-(carbamoylmethyl)-5,15-dichloro-2,18,32,35,37-pentahydroxy-19-[(2R)-4-methyl-2-(methylamino)pentanamido]-20,23,26,42,44-pentaoxo-7,13-dioxa-21,24,27,41,43-pentaazaoctacyclo[26.14.2.2^{3,6}.2^{14,17}.1^{8,12}.1^{29,33}.0^{10,25}.0^{34,39}]pentaconta-3,5,8(48),9,11,14,16,29(45),30,32,34,36,38,46,49-pentadecaene-40-carboxylic acid);

or derivatives, salts, or analogues thereof, or combinations thereof.

As used herein, an “immunomodulatory compound” has its ordinary meaning as understood in light of the specification and refers to a compound that modulates an immune response in a subject. An immunomodulatory compound may include, for example:

Cyclosporin A (including pharmaceutically acceptable salts thereof. Cyclosporin A includes CsA, ciclosporin A, cyclosporine A, Neoral, Sandimmune, (3S,6S,9S,12R,15S,18S,21S,24S,30S,33S)-30-Ethyl-33-[(1R,2R,4E)-1-hydroxy-2-methyl-4-hexen-1-yl]-6,9,18,24-tetraisobutyl-3,21-diisopropyl-1,4,7,10,12,15,19,25,28-nonamethyl-1,4,7,10,13,16,19,22,25,28,31-undecaazacyclotritriacontane-2,5,8,11,14,17,20,23,26,29,32-undecone);

An inhibitor of the NLRP3 inflammasome. An inhibitor of NLRP3 inflammasome is also referred to herein as an NLRP3 antagonist or an NLRP3 inhibitor. NLRP3, or nucleotide-binding oligomerization domain (NOD) like receptor (NLR) pyrin domain-containing protein 3 inflammasome is an innate immune sensor that upon assembly activates caspase-1 and mediates the processing and release of IL-1(3. Exemplary NLRP3 inhibitors include, for example, Ac-YVAD-cmk, 2-APB, arglabin, BAPTA, BAY 11-7082, (3-hydroxybutyrate (BHB), C172, CY-09, flufenamic acid, glybenclamide, INF39, isoliquiritigenin, MCC950, mefenamic acid, 3,4-methylenedioxy-β-nitrostyrene (MNS), OLT1177, oridonin, parthenolide, resveratrol, sulforaphane, tranilast, VX-765, and Z-VAD-FMK;

Ac-YVAD-cmk (including pharmaceutically acceptable salts thereof. Ac-YVAD-cmk includes chloromethyl ketone tetrapeptide based on the target sequence in proIL-1β YVHD, N-acetyl-tyrosyl-valyl-alanyl-aspartyl chloromethyl ketone)

2-APB (including pharmaceutically acceptable salts thereof. 2-APB includes 2-aminoethoxydiphenyl borate, 2-diphenylboranyloxyethanamine);

Arglabin (including pharmaceutically acceptable salts thereof. Arglabin includes (3aR,4aS,6aS,9aS,9bR)-1,4a-Dimethyl-7-methylene-5,6,6a,7,9a,9b-hexahydro-3H-oxireno[8,8a]azuleno[4,5-b]furan-8(4aH)-one);

BAPTA (including pharmaceutically acceptable salts thereof. BAPTA includes 1,2-bis(o-aminophenoxy)ethane-N,N,N′,N′-tetraacetic acid);

BAY 11-7082 (including pharmaceutically acceptable salts thereof. BAY 11-7082 includes (E)-3-tosylacrylonitrile);

BHB (including pharmaceutically acceptable salts thereof. BHB includes β-hydroxybutyrate, 3-hydroxybutyrate);

C172 (including pharmaceutically acceptable salts thereof. C172 includes CFTR₍ᵢₙₕ₎-172, (Z)-4-((4-oxo-2-thioxo-3-(5-(trifluoromethyl)phenyl)thiazolidin-5-ylidene)methyl)benzoic acid);

CY-09 (including pharmaceutically acceptable salts thereof. CY-09 includes (Z)-4-((4-oxo-2-thioxo-3-(3-(trifluoromethyl)phenyl)thiazolidin-5-ylidene)methyl)benzoic acid)

Flufenamic acid (including pharmaceutically acceptable salts thereof. Flufenamic acid includes Flufenerim, Flufenoxuron, Flufenisal, 2-((3-(trifluoromethyl)phenyl)amino)benzoic acid);

Glybenclamide (including pharmaceutically acceptable salts thereof. Glybenclamide includes glibenclamide, glyburide, Micronase, Maninil, 5-chloro-N-[2-[4-(cyclohexylcarbamoylsulfamoyl)phenyl]ethyl]-2-methoxybenzamide);

INF39 (including pharmaceutically acceptable salts thereof. INF39 includes ethyl 2-(2-chlorobenzyl)acrylate);

Isoliquiritigenin (including pharmaceutically acceptable salts thereof. Isoliquiritigenin includes (E)-1-(2,4-Dihydroxyphenyl)-3-(4-hydroxyphenyl)prop-2-en-1-one);

MCC950 (including pharmaceutically acceptable salts thereof. MCC950 includes N-((1,2,3,5,6,7-hexahydro-s-indacen-4-yl)carbamoyl)-4-(2-hydroxypopan-2-yl)furan-2-sulfonamide);

Mefenamic acid (including pharmaceutically acceptable salts thereof. Mefenamic acid includes Ponstel, Ponstan, 2-(2,3-dimethylphenyl)aminobenzoic acid);

3,4-methylenedioxy-β-nitrostyrene (MNS) (including pharmaceutically acceptable salts thereof);

OLT1177 (including pharmaceutically acceptable salts thereof. OLT1177 includes 3-(methylsulfonyl)-propanenitrile);

Oridonin (including pharmaceutically acceptable salts thereof. Oridonin includes Isodonol, 7a,20-Epoxy-1a,6b,7,14-tetrahydroxy-Kaur-16-en-15-one);

Parthenolide (including pharmaceutically acceptable salts thereof. Parthenolide includes (3 aS,9aR,10aS,10bS,E)-6,9a-dimethyl-3-methylene-3a,4,5,8,9,9a,10a,10b-octahydrooxireno[2′,3′: 9,10]cyclodeca[1,2-b]furan-2(3H)-one);

Resveratrol (including pharmaceutically acceptable salts thereof. Resveratrol includes trans-3,5,4′-Trihydroxystilbene, 3,4′,5-Stilbenetriol, trans-Resveratrol, (E)-5-(p-Hydroxystyryl)resorcinol, (E)-5-(4-hydroxystyryl)benzene-1,3-diol);

Sulforaphane (including pharmaceutically acceptable salts thereof. Sulforaphane includes 1-Isothiocyanato-4-methylsulfinylbutane);

Tranilast (including pharmaceutically acceptable salts thereof. Tranilast includes Rizaben, 2-{[(2E)-3-(3,4-Dimethoxyphenyl)prop-2-enoyl]amino}benzoic acid);

VX-765 (including pharmaceutically acceptable salts thereof. VX-765 includes (S)-1-((S)-2-{[1-(4-amino-3-chloro-phenyl)-methanoyl]-amino}-3,3-dimethyl-butanoyl)-pyrrolidine-2-carboxylic acid ((2R,3S)-2-ethoxy-5-oxo-tetrahydro-furan-3-yl)-amide);

Z-VAD-FMK (including pharmaceutically acceptable salts thereof. Z-VAD-FMK includes carbobenzoxy-valyl-alanyl-aspartyl-[O-methyl]-fluoromethylketone).

Additional immunomodulatory compounds may include, for example:

Diclofenac (including pharmaceutically acceptable salts thereof. Diclofenac includes Cataflam, Voltaren, [2-(2,6-Dichloroanilino)phenyl]acetic acid);

Ketorolac (including pharmaceutically acceptable salts thereof. Ketorolac includes Toradol, Acular, Spric, ketorolac tromethamine, (±)-5-benzoyl-2,3-dihydro-1H-pyrrolizine-1-carboxylic acid);

Bromfenac (including pharmaceutically acceptable salts thereof. Bromfenac includes Bromday, Prolensa, Yellox, 2-[2-amino-3-(4-bromobenzoyl)phenyl]acetic acid);

Nepafenac (including pharmaceutically acceptable salts thereof. Nepafenac includes Amnac, Ilevro, 2-amino-3-benzoylbenzeneacetamide);

Flurbiprofen (including pharmaceutically acceptable salts thereof. Flurbiprofen includes Ansaid, Ocufen, Strepfen, (±)-2-fluoro-α-methyl-(1,1′-biphenyl) acetic acid, (RS)-2-(2-fluorobiphenyl-4-yl)propanoic acid);

Lifitegrast (including pharmaceutically acceptable salts thereof. Lifitegrast includes Xiidra, N-{[2-(1-Benzofuran-6-ylcarbonyl)-5,7-dichloro-1,2,3,4-tetrahydro-6-isoquinolinyl]carbonyl}-3-(methylsulfonyl)-L-phenylalanine);

or derivatives, salts, or analogues thereof, or combinations thereof.

As used herein, a “steroid” has its ordinary meaning as understood in light of the specification and refers to naturally occurring steroids and their derivatives as well as synthetic or semi-synthetic steroid analogues having steroid-like activity. The steroid can be a glucocorticoid or corticosteroid. A steroid may include, for example:

Dexamethasone (including pharmaceutically acceptable salts thereof. Dexamethasone includes (8S,9R,10S,11S,13S,14S,16R,17R)-9-Fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one);

Difluprednate (including pharmaceutically acceptable salts thereof. Difluprednate includes [(6S,8S,9R,10S,11S,13S,14S,17R)-17-(2-acetyloxyacetyl)-6,9-difluoro-11-hydroxy-10,13-dimethyl-3-oxo-6,7,8,11,12,14,15,16-octahydrocyclopenta[a]phenanthren-17-yl]butanoate);

Fluorometholone (including pharmaceutically acceptable salts thereof. Fluorometholone includes Efflumidex, Flucon, FML Forte, FML, (6S,8S,9R,10S,11S,13S,14S,17R)-17-acetyl-9-fluoro-11,17-dihydroxy-6,10,13-trimethyl-6,7,8,11,12,14,15,16-octahydrocyclopenta[a]phenanthren-3-one, (1R,2S,8S,10S,11S,14R,15S,17S)-14-acetyl-1-fluoro-14,17-dihydroxy-2,8,15-trimethyltetracyclo[8.7.0.0^{2,7}.0^{11,15}]heptadeca-3,6-dien-5-one);

Loteprednol (including pharmaceutically acceptable salts thereof. Loteprednol includes Lotemax, 11β,17α,Dihydroxy-21-oxa-21-chloromethylpregna-1,4-diene-3,20-dione 17α-ethylcarbonate, Chloromethyl 17-ethoxycarbonyloxy-11-hydroxy-10,13-dimethyl-3-oxo-7,8,9,11,12,14,15,16-octahydro-6H-cyclopenta[a]phenanthrene carboxylate);

Prednisolone (including pharmaceutically acceptable salts thereof. Prednisolone includes 11,17-Dihydroxy-17-(2-hydroxyacetyl)-10,13-dimethyl-6,7,8,9,10,11,12, 13,14,15,16,17-dodecahydrocyclopenta[a]phenanthren-3-one, (11(3)-11,17,21-Trihydroxypregna-1,4-diene-3,20-dione);

Rimexolone (including pharmaceutically acceptable salts thereof. Rimexolone includes Vexol, Trimexolone, Org 6216, 11β-Hydroxy-16a,17a,21-trimethylpregna-1,4-dien-3,20-dione, (8S,9S,10R,11S,13S,14S,16R,17S)-11-Hydroxy-10,13,16,17-tetramethyl-17-propanoyl-7,8,9,11,12,14,15,16-octahydro-6H-cyclopenta[a]phenanthren-3-one);

or derivatives, salts, or analogues thereof, or combinations thereof.

In some embodiments, the one or more additional therapeutic agent is oxymetazoline. In some embodiments, the one or more additional therapeutic agent is brimonidine.

In some embodiments, the one or more RNase comprises a single RNase, and the one or more additional therapeutic agent comprises a single additional therapeutic agent. In such embodiments, any single RNase disclosed herein may be combined in the product combination with any single additional therapeutic agent disclosed herein.

In some embodiments, the one or more RNase comprises a plurality of RNases and the one or more additional therapeutic agent comprises a single additional therapeutic agent. In such embodiments, any plurality of RNases disclosed herein may be combined in the product combination with any single additional therapeutic agent disclosed herein.

In some embodiments, the one or more RNase comprises a single RNase, and the one or more additional therapeutic agent comprises a plurality or additional therapeutic agents. In such embodiments, any single RNase disclosed herein may be combined in the product combination with any plurality of additional therapeutic agents disclosed herein.

In some embodiments, the one or more RNase comprises a plurality of RNases, and the one or more additional therapeutic agent comprises a plurality of additional therapeutic agents. In such embodiments, any plurality of RNases disclosed herein may be combined in the product combination with any plurality of additional therapeutic agents disclosed herein.

In some embodiments, the one or more RNase comprises 1, 2, 3, 4, 5, 6, 7, 8, 9, 10, 11, 12, 13, 14, or 15 or more RNases as disclosed herein. In some embodiments, the one or more RNase comprises at least 1, at least 2, at least 3, at least 4, at least 5, at least 6, at least 7, at least 8, at least 9, at least 10, at least 11, at least 12, at least 13, at least 14, or at least 15 RNases as disclosed herein. In some embodiments, the one or more RNase comprises at most 1, at most 2, at most 3, at most 4, at most 5, at most 6, at most 7, at most 8, at most 9, at most 10, at most 11, at most 12, at most 13, at most 14, or at most 15 RNases as disclosed herein. In some embodiments, the one or more RNase comprises 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 2 to 3, 2 to 4, 2 to 5, 2 to 6, 2 to 7, 2 to 8, 2 to 9, 2 to 10, 2 to 11, 2 to 12, 2 to 13, 2 to 14, 2 to 15, 3 to 4, 3 to 5, 3 to 6, 3 to 7, 3 to 8, 3 to 9, 3 to 10, 3 to 11, 3 to 12, 3 to 13, 3 to 14, 3 to 15, 4 to 5, 4 to 6, 4 to 7, 4 to 8, 4 to 9, 4 to 10, 4 to 11, 4 to 12, 4 to 13, 4 to 14, 4 to 15, 5 to 6, 5 to 7, 5 to 8, 5 to 9, 5 to 10, 5 to 11, 5 to 12, 5 to 13, 5 to 14, 5 to 15, 6 to 7, 6 to 8, 6 to 9, 6 to 10, 6 to 11, 6 to 12, 6 to 13, 6 to 14, 6 to 15, 7 to 8, 7 to 9, 7 to 10, 7 to 11, 7 to 12, 7 to 13, 7 to 14, 7 to 15, 8 to 9, 8 to 10, 8 to 11, 8 to 12, 8 to 13, 8 to 14, 8 to 15, 9 to 10, 9 to 11, 9 to 12, 9 to 13, 9 to 14, 9 to 15, 10 to 11, 10 to 12, 10 to 13, 10 to 14, 10 to 15, 11 to 12, 11 to 13, 11 to 14, 11 to 15, 12 to 13, 12 to 14, 12 to 15, 13 to 14, 13 to 15, or 14-15 RNases as disclosed herein.

In some embodiments, the one or more additional therapeutic agent comprises 1, 2, 3, 4, 5, 6, 7, 8, 9, 10, 11, 12, 13, 14, or 15 or more additional therapeutic agents as disclosed herein. In some embodiments, the one or more additional therapeutic agent comprises at least 1, at least 2, at least 3, at least 4, at least 5, at least 6, at least 7, at least 8, at least 9, at least 10, at least 11, at least 12, at least 13, at least 14, or at least 15 additional therapeutic agents as disclosed herein. In some embodiments, the one or more additional therapeutic agent comprises at most 1, at most 2, at most 3, at most 4, at most 5, at most 6, at most 7, at most 8, at most 9, at most 10, at most 11, at most 12, at most 13, at most 14, or at most 15 additional therapeutic agents as disclosed herein. In some embodiments, the one or more additional therapeutic agent comprises 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15,2 to 3,2 to 4,2 to 5, 2 to 6, 2 to 7, 2 to 8, 2 to 9, 2 to 10, 2 to 11, 2 to 12, 2 to 13, 2 to 14, 2 to 15, 3 to 4, 3 to 5, 3 to 6, 3 to 7, 3 to 8, 3 to 9, 3 to 10, 3 to 11, 3 to 12, 3 to 13, 3 to 14, 3 to 15, 4 to 5, 4 to 6, 4 to 7, 4 to 8, 4 to 9, 4 to 10, 4 to 11, 4 to 12, 4 to 13, 4 to 14, 4 to 15, 5 to 6, 5 to 7, 5 to 8, 5 to 9, 5 to 10, 5 to 11, 5 to 12, 5 to 13, 5 to 14, 5 to 15, 6 to 7, 6 to 8, 6 to 9, 6 to 10, 6 to 11, 6 to 12, 6 to 13, 6 to 14, 6 to 15, 7 to 8, 7 to 9, 7 to 10, 7 to 11, 7 to 12, 7 to 13, 7 to 14, 7 to 15, 8 to 9, 8 to 10, 8 to 11, 8 to 12, 8 to 13, 8 to 14, 8 to 15, 9 to 10, 9 to 11, 9 to 12, 9 to 13, 9 to 14, 9 to 15, 10 to 11, 10 to 12, 10 to 13, 10 to 14, 10 to 15, 11 to 12, 11 to 13, 11 to 14, 11 to 15, 12 to 13, 12 to 14, 12 to 15, 13 to 14, 13 to 15, or 14-15 additional therapeutic agents as disclosed herein.

In any of the embodiments described herein, any combination of RNases may be combined with any combination of additional therapeutic agents for the formulation of the product combination.

Some embodiments provided herein relate to product combinations that includes one or more RNase as disclosed herein in combination with one or more additional therapeutic agent, such as one or more vasoconstrictor, antibiotic, immunomodulatory compound, or steroid, as described herein. In one embodiment, a product combination includes a composition comprising ranpirnase, or a variant, analogue, or fraction thereof and a composition comprising one or more additional therapeutic agent, such as one or more vasoconstrictor, antibiotic, immunomodulatory compound, or steroid, as described herein. In one embodiment, a product combination includes a composition comprising amphinase, or a variant, analogue, or fraction thereof and a composition comprising one or more additional therapeutic agent, such as one or more vasoconstrictor, antibiotic, immunomodulatory compound, or steroid, as described herein.

In some embodiments, the product combination comprises any ranpirnase described herein, including any variant, analogue, derivative, or fraction thereof and one or more of naphazoline, tetrahydrozoline, phenylephrine, oxymetazoline, brimonidine, apraclonidine, ephedrine, azithromycin, erythromycin, gentamicin, neomycin, tobramycin, besifloxacin, ciprofloxacin, gatifloxacin, levofloxacin, moxifloxacin, ofloxacin, bacitracin, chloramphenicol, gramicidin, natamycin, polymyxin B, sulfacetamide, tetracycline, trimethoprim, vancomycin, dexamethasone, difluprednate, fluorometholone, loteprednol, prednisolone, rimexolone, cyclosporine A, an NLRP3 inhibitor, diclofenac, ketorolac, bromfenac, nepafenac, flurbiprofen, lifitegrast, or a pharmaceutically acceptable salt, analogue, or derivative thereof. Inhibitors of NLRP3 include, for example, Ac-YVAD-cmk, 2-APB, arglabin, BAPTA, BAY 11-7082, β-hydroxybutyrate (BHB), C172, CY-09, flufenamic acid, glybenclamide, INF39, isoliquiritigenin, MCC950, mefenamic acid, 3,4-methylenedioxy-β-nitrostyrene (MNS), OLT1177, oridonin, parthenolide, resveratrol, sulforaphane, tranilast, VX-765, or Z-VAD-FMK.

In some embodiments, the product combination comprises any amphinase described herein, including any variant, analogue, derivative, or fraction thereof and one or more of naphazoline, tetrahydrozoline, phenylephrine, oxymetazoline, brimonidine, apraclonidine, ephedrine, azithromycin, erythromycin, gentamicin, neomycin, tobramycin, besifloxacin, ciprofloxacin, gatifloxacin, levofloxacin, moxifloxacin, ofloxacin, bacitracin, chloramphenicol, gramicidin, natamycin, polymyxin B, sulfacetamide, tetracycline, trimethoprim, vancomycin, dexamethasone, difluprednate, fluorometholone, loteprednol, prednisolone, rimexolone, cyclosporine A, an NLRP3 inhibitor, diclofenac, ketorolac, bromfenac, nepafenac, flurbiprofen, lifitegrast, or a pharmaceutically acceptable salt, analogue, or derivative thereof. Inhibitors of NLRP3 include, for example, Ac-YVAD-cmk, 2-APB, arglabin, BAPTA, BAY 11-7082, β-hydroxybutyrate (BHB), C172, CY-09, flufenamic acid, glybenclamide, INF39, isoliquiritigenin, MCC950, mefenamic acid, 3,4-methylenedioxy-β-nitrostyrene (MNS), OLT1177, oridonin, parthenolide, resveratrol, sulforaphane, tranilast, VX-765, or Z-VAD-FMK.

In some embodiments, the product combination includes ranpirnase in combination with oxymetazoline. In some embodiments, the product combination includes ranpirnase in combination with brimonidine.

In some embodiments, the one or more RNase, or a pharmaceutically acceptable salt thereof, is prepared in a composition, and the one or more additional therapeutic agent is prepared in a composition, and each composition is separate from the other. In some embodiments, the separate compositions are prepared for administration in combination. The order of administration of the composition comprising an RNase, or a pharmaceutically acceptable salt thereof, with the composition comprising one or more additional therapeutic agent(s) can vary. In some embodiments, a composition comprising an RNase, or a pharmaceutically acceptable salt thereof, can be administered prior to all additional therapeutic agents. In other embodiments, a composition comprising an RNase, or a pharmaceutically acceptable salt thereof, can be administered prior to at least one additional therapeutic agent. In still other embodiments, a composition comprising an RNase, or a pharmaceutically acceptable salt thereof, can be administered concomitantly with one or more additional therapeutic agents. In yet still other embodiments, a composition comprising an RNase, or a pharmaceutically acceptable salt thereof, can be administered subsequent to the administration of at least one additional therapeutic agent. In some embodiments, a composition comprising an RNase, or a pharmaceutically acceptable salt thereof, can be administered subsequent to the administration of all additional therapeutic agents.

Some embodiments provided herein comprise a kit comprising one or more RNase, or a pharmaceutically acceptable salt thereof and one or more additional therapeutic agents. In some embodiments, the kit further comprises instructions for administering the compositions, including instructions whether to administer the compositions in sequence or in parallel when the compositions are prepared separately, and instructions for administering a single formulation when the product combination is prepared as a single dosage formulation. In some embodiments, one or more RNase, or a pharmaceutically acceptable salt thereof is provided in a first container and one or more additional therapeutic agents is provided in a second container.

In some embodiments, the one or more RNase, or a pharmaceutically acceptable salt thereof, and the one or more additional therapeutic agents are formulated in a single formulation or in a single dosage.

### III. Dosage Formulations

Embodiments of the product combination comprising one or more RNase, variants, derivatives, analogues, or a pharmaceutically acceptable salt thereof one or more additional therapeutic agents or derivatives, analogues, or salts thereof are formulated as a product combination for administration to a subject using a cellular uptake approach. In some embodiments, the one or more RNase is prepared as a first pharmaceutical composition and the one or more additional therapeutic agent is prepared as a second pharmaceutical composition. In some embodiments, the first and second pharmaceutical compositions are used in combination as a product combination.

In some embodiments, the product combination is a single formulation that includes one or more RNase, variants, derivatives, analogues, or pharmaceutically acceptable salts thereof and one or more additional therapeutic agent, or derivatives, analogues, or salts thereof.

A pharmaceutical composition disclosed herein may optionally include a pharmaceutically acceptable carrier that facilitates processing of an active ingredient into pharmaceutically acceptable compositions. As used herein, the term “pharmacologically-acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures, and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening, or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride (BAK), chlorobutanol, thimerosal, phenylmercuric acetate, phenylmercuric nitrate, a stabilized oxy chloro composition, such as, e.g., PURITE® and chelants, such as, e.g., DTPA or DTPA-bisamide, calcium DTPA, and CaNaDTPA-bisamide. Many of these preservatives have bactericidal properties. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

A pharmaceutical composition disclosed herein may be formulated for either local or systemic delivery using topical, ophthalmic, enteral, or parenteral routes of administration. In addition, a pharmaceutical composition disclosed herein may be produced as a liquid formulation, a semi-solid formulation, or a solid formulation. A formulation disclosed herein can be produced in a manner to form one phase, such as, e.g., an oil or a solid. Alternatively, a formulation disclosed herein can be produced in a manner to form two phases, such as, e.g., a colloidal formulation. A pharmaceutical composition disclosed herein intended for such administration may be prepared according to any method known to the art for the manufacture of pharmaceutical compositions. Liquid formulations suitable for topical and ophthalmologic administration include, without limitation, solutions and emulsions. Semi-solid formulations suitable for topical and ophthalmologic administration include, without limitation, ointments, creams, salves, foams, and gels. Solid formulations suitable for topical and ophthalmologic administration include, without limitation, gel implants, solid sol implants and solid implants.

The amount of one or more RNase in the product combination and one or more additional therapeutic agent in the product combination is a therapeutically effective amount, for example, an amount sufficient to reduce, prevent, inhibit, treat, mitigate, ameliorate, or slow a viral replication or infection, including symptoms of a viral infection. A therapeutically effective amount is an amount that does not cause significant adverse side effects. Such amount may vary depending on which specific RNase is administered and which specific additional therapeutic agent is administered, in addition to quantities of a specific RNase and a specific additional therapeutic agent in compatible amounts in a product combination. An optimal amount for a particular product combination can be ascertained by standard studies involving observation of proinflammatory cytokine titers, anti-inflammatory cytokine titers, prostaglandin titers, reduction of one or more symptoms associated with a viral conjunctivitis, and other responses in individuals. A primary pharmaceutical composition course may include 1, 2, 3 or 4 doses of a pharmaceutical composition, given at intervals optimal for providing an anti-inflammatory response.

Generally, an effective and safe amount of an RNase or of an additional therapeutic agent in a product combination is in a range from about 1 fg to about 3,000 mg. In some embodiments, an amount of a RNase or of an additional therapeutic agent disclosed herein included in a product combination may be separately about 1 fg, about 2 fg, about 3 fg, about 4 fg, about 5 fg, about 6 fg, about 7 fg, about 8 fg, about 9 fg, about 10 fg, about 15 fg, about 20 fg, about 25 fg, about 30 fg, about 35 fg, about 40 fg, about 45 fg, about 50 fg, about 55 fg, about 60 fg, about 65 fg, about 70 fg, about 75 fg, about 80 fg, about 85 fg, about 90 fg, about 95 fg, about 100 fg, about 110 fg, about 120 fg, about 130 fg, about 140 fg, about 150 fg, about 160 fg, about 170 fg, about 180 fg, about 190 fg, about 200 fg, about 210 fg, about 220 fg, about 230 fg, about 240 fg, about 250 fg, 260 fg, about 270 fg, about 280 fg, about 290 fg, about 300 fg, about 310 fg, about 320 fg, about 330 fg, about 340 fg, about 350 fg, 360 fg, about 370 fg, about 380 fg, about 390 fg, about 400 fg, about 410 fg, about 420 fg, about 430 fg, about 440 fg, about 450 fg, 460 fg, about 470 fg, about 480 fg, about 490 fg, about 500 fg, about 510 fg, about 520 fg, about 530 fg, about 540 fg, about 550 fg, 560 fg, about 570 fg, about 580 fg, about 590 fg, about 600 fg, about 610 fg, about 620 fg, about 630 fg, about 640 fg, about 650 fg, 660 fg, about 670 fg, about 680 fg, about 690 fg, about 700 fg, about 710 fg, about 720 fg, about 730 fg, about 740 fg, about 750 fg, 760 fg, about 770 fg, about 780 fg, about 790 fg, about 800 fg, about 810 fg, about 820 fg, about 830 fg, about 840 fg, about 850 fg, 860 fg, about 870 fg, about 880 fg, about 890 fg, about 900 fg, about 910 fg, about 920 fg, about 930 fg, about 940 fg, about 950 fg, 960 fg, about 970 fg, about 980 fg, about 990 fg, or about 1,000 fg, or in an amount within a range defined by any two of the aforementioned values.

In some embodiments, an amount of a RNase or of an additional therapeutic agent disclosed herein included in a product combination may be separately about 1 ng, about 2 ng, about 3 ng, about 4 ng, about 5 ng, about 6 ng, about 7 ng, about 8 ng, about 9 ng, about 10 ng, about 15 ng, about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 110 ng, about 120 ng, about 130 ng, about 140 ng, about 150 ng, about 160 ng, about 170 ng, about 180 ng, about 190 ng, about 200 ng, about 210 ng, about 220 ng, about 230 ng, about 240 ng, about 250 ng, 260 ng, about 270 ng, about 280 ng, about 290 ng, about 300 ng, about 310 ng, about 320 ng, about 330 ng, about 340 ng, about 350 ng, 360 ng, about 370 ng, about 380 ng, about 390 ng, about 400 ng, about 410 ng, about 420 ng, about 430 ng, about 440 ng, about 450 ng, 460 ng, about 470 ng, about 480 ng, about 490 ng, about 500 ng, about 510 ng, about 520 ng, about 530 ng, about 540 ng, about 550 ng, 560 ng, about 570 ng, about 580 ng, about 590 ng, about 600 ng, about 610 ng, about 620 ng, about 630 ng, about 640 ng, about 650 ng, 660 ng, about 670 ng, about 680 ng, about 690 ng, about 700 ng, about 710 ng, about 720 ng, about 730 ng, about 740 ng, about 750 ng, 760 ng, about 770 ng, about 780 ng, about 790 ng, about 800 ng, about 810 ng, about 820 ng, about 830 ng, about 840 ng, about 850 ng, 860 ng, about 870 ng, about 880 ng, about 890 ng, about 900 ng, about 910 ng, about 920 ng, about 930 ng, about 940 ng, about 950 ng, 960 ng, about 970 ng, about 980 ng, about 990 ng, or about 1,000 ng, or in an amount within a range defined by any two of the aforementioned values.

In some embodiments, an amount of a RNase or of an additional therapeutic agent disclosed herein included in a product combination may be separately about 1 μg, about 2 μg, about 3 μg, about 4 μg, about 5 μg, about 6 μg, about 7 μg, about 8 μg, about 9 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, 260 μg, about 270 μg, about 280 μg, about 290 μg, about 300 μg, about 310 μg, about 320 μg, about 330 μg, about 340 μg, about 350 μg, 360 μg, about 370 μg, about 380 μg, about 390 μg, about 400 μg, about 410 μg, about 420 μg, about 430 μg, about 440 μg, about 450 μg, 460 μg, about 470 μg, about 480 μg, about 490 μg, about 500 μg, about 510 μg, about 520 μg, about 530 μg, about 540 μg, about 550 μg, 560 μg, about 570 μg, about 580 μg, about 590 μg, about 600 μg, about 610 μg, about 620 μg, about 630 μg, about 640 μg, about 650 μg, 660 μg, about 670 μg, about 680 μg, about 690 μg, about 700 μg, about 710 μg, about 720 μg, about 730 μg, about 740 μg, about 750 μg, 760 μg, about 770 μg, about 780 μg, about 790 μg, about 800 μg, about 810 μg, about 820 μg, about 830 μg, about 840 μg, about 850 μg, 860 μg, about 870 μg, about 880 μg, about 890 μg, about 900 μg, about 910 μg, about 920 μg, about 930 μg, about 940 μg, about 950 μg, 960 μg, about 970 μg, about 980 μg, about 990 μg, or about 1,000 μg, or in an amount within a range defined by any two of the aforementioned values.

In some embodiments, an amount of a RNase or of an additional therapeutic agent disclosed herein included in a product combination may be separately about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, 260 mg, about 270 mg, about 280 mg, about 290 mg, about 300 mg, about 310 mg, about 320 mg, about 330 mg, about 340 mg, about 350 mg, 360 mg, about 370 mg, about 380 mg, about 390 mg, about 400 mg, about 410 mg, about 420 mg, about 430 mg, about 440 mg, about 450 mg, 460 mg, about 470 mg, about 480 mg, about 490 mg, about 500 mg, about 510 mg, about 520 mg, about 530 mg, about 540 mg, about 550 mg, 560 mg, about 570 mg, about 580 mg, about 590 mg, about 600 mg, about 610 mg, about 620 mg, about 630 mg, about 640 mg, about 650 mg, 660 mg, about 670 mg, about 680 mg, about 690 mg, about 700 mg, about 710 mg, about 720 mg, about 730 mg, about 740 mg, about 750 mg, 760 mg, about 770 mg, about 780 mg, about 790 mg, about 800 mg, about 810 mg, about 820 mg, about 830 mg, about 840 mg, about 850 mg, 860 mg, about 870 mg, about 880 mg, about 890 mg, about 900 mg, about 910 mg, about 920 mg, about 930 mg, about 940 mg, about 950 mg, 960 mg, about 970 mg, about 980 mg, about 990 mg, about 1,000 mg, about 1,250 mg, about 1,500 mg, about 1,750 mg, about 2,000 mg, about 2,250 mg, about 2,500 mg, about 2,750 mg, or about 3,000 mg, or in an amount within a range defined by any two of the aforementioned values.

In some embodiments, the amount of ranpirnase present in the product combinations described herein is expressed in terms of percent weight volume (% w/v) or in terms of molarity (M). In some embodiments, the ranpirnase is present in an amount ranging from about 0.001% to about 5% w/v, such as 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% w/v or in an amount within a range defined by any two of the aforementioned values. In some embodiments, the ranpirnase is present in a range of about 0.001% to about 1% w/v, 0.01% to about 1% w/v, 0.001% to about 0.1% w/v, about 0.01% to about 0.1% w/v, about 0.005% to about 5% w/v, 0.05% to about 1% w/v, 0.005% to about 0.5% w/v, about 0.05% to about 0.05% w/v, about 0.02% to about 0.05% w/v, or about 0.02% to about 0.04% w/v, or any range defined by any two of the aforementioned values. In some embodiments, the ranpirnase is present in an amount ranging from about 1 μM to about 4 M, such as 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, or 1000 μM, or 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1000, 1500, 2000, 2500, 3000, 3500, or 4000 mM, or in an amount within a range defined by any two of the aforementioned values. In some embodiments the ranpirnase is present in a range of about 1 μM to about 30 μM, 10 μM to about 30 μM, 1 mM to about 30 mM, or 20 mM to about 30 mM.

In some embodiments, the amount of one or more additional therapeutic agent in the product combinations described herein is expressed in terms of % w/v or in terms of M. In some embodiments, the one or more additional therapeutic agent is present in an amount of 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5%, w/v or in an amount within a range defined by any two of the aforementioned values. In some embodiments, the one or more additional therapeutic is present in a range of about 0.001% to about 1% w/v, 0.01% to about 1% w/v, 0.001% to about 0.1% w/v, about 0.01% to about 0.1% w/v, about 0.005% to about 5% w/v, 0.05% to about 1% w/v, 0.005% to about 0.5% w/v, about 0.05% to about 0.05% w/v, about 0.02% to about 0.05% w/v, or about 0.02% to about 0.04% w/v, or any range defined by any two of the aforementioned values. In some embodiments, the one or more additional therapeutic agent is present in an amount ranging from about 1 μM to about 4 M, such as 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, or 1000 μM, or 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1000, 1500, 2000, 2500, 3000, 3500, or 4000 mM, or in an amount within a range defined by any two of the aforementioned values. In some embodiments the ranpirnase is present in a range of about 1 μM to about 30 μM, 10 μM to about 30 μM, 1 mM to about 30 mM, or 20 mM to about 30 mM.

In some embodiments, the product combination includes ranpirnase present in any amount as described herein, and oxymetazoline present in any amount described herein with respect to the one or more additional therapeutic agents. In some embodiments, the product combination includes ranpirnase present in an amount of about 0.001% to about 5% w/v and oxymetazoline present in an amount of about 0.001% to about 5% w/v. In some embodiments, the product combination includes ranpirnase present in an amount of about 0.01% to about 0.05% w/v and oxymetazoline present in an amount of about 0.01% to about 0.05% w/v. In some embodiments, the product combination includes ranpirnase present in an amount of about 0.02% to about 0.04% w/v and oxymetazoline present in an amount of about 0.02% to about 0.03% w/v. In some embodiments, the product combination includes ranpirnase present in an amount of about 0.03% w/v and oxymetazoline present in an amount of about 0.01% to about 0.025% w/v.

In some embodiments, the product combination includes ranpirnase present in any amount as described herein, and brimonidine present in any amount described herein with respect to the one or more additional therapeutic agents. In some embodiments, the product combination includes ranpirnase present in an amount of about 0.001% to about 5% w/v and brimonidine present in an amount of about 0.001% to about 5% w/v. In some embodiments, the product combination includes ranpirnase present in an amount of about 0.01% to about 0.05% w/v and brimonidine present in an amount of about 0.01% to about 0.05% w/v. In some embodiments, the product combination includes ranpirnase present in an amount of about 0.02% to about 0.04% w/v and brimonidine present in an amount of about 0.02% to about 0.03% w/v. In some embodiments, the product combination includes ranpirnase present in an amount of about 0.03% w/v and brimonidine present in an amount of about 0.01% to about 0.025% w/v.

The product combination comprising one or more RNase and the one or more additional therapeutic agent as disclosed herein may be formulated in a controlled release delivery platform including a sustained release formulation and an extended-release formulation. The ocular surface is a challenging target tissue for administration of a drug because tear production immediately dilutes active ingredient upon administration. Further, blinking dilutes and removes active ingredient being administered. The use of a controlled release delivery platform adheres of the ocular surface to ensure that one or more RNase and one or more additional therapeutic agent disclosed herein remains for a time sufficient to deliver the required dose necessary for therapeutic effect. Such controlled release delivery platform can improve the delivery kinetics of the one or more RNase and the one or more additional therapeutic agent disclosed herein by releasing in a time-controlled fashion, potentially minimizing the number of instillations required over a course of treatment.

An extended-release formulation refers to the release of one or more RNase disclosed herein and/or one or more additional therapeutic agent disclosed herein over a period of time of less than about seven days. A sustained release formulation refers to the release of one or more RNase disclosed herein and/or one or more additional therapeutic agent disclosed herein over a period of about seven days or more.

In some embodiments, a sustained release formulation releases one or more RNase disclosed herein and/or one or more additional therapeutic agent with substantially zero order release kinetics over a period of, e.g., about 7 days, about 15 days after administration, about 30 days, about 45 days, about 60 days, about 75 days, or about 90 days after administration. In some embodiments, a sustained release formulation releases one or more RNase disclosed herein and/or one or more additional therapeutic agent with substantially first order release kinetics over a period of, e.g., about 7 days, about 15 days after administration, about 30 days, about 45 days, about 60 days, about 75 days, or about 90 days after administration.

In some embodiments, an extended-release formulation releases one or more RNase disclosed herein and/or one or more additional therapeutic agent with substantially zero order release kinetics over a period of, e.g., about 1 day, about 2 days, about 3 days, about 4 days, about 5 days, about 6 days, or about 7 days after administration. In some embodiments, an extended-release formulation releases one or more RNase disclosed herein and/or one or more additional therapeutic agent with substantially first order release kinetics over a period of, e.g., about 1 day, about 2 days, about 3 days, about 4 days, about 5 days, about 6 days, or about 7 days after administration.

Dosing can be single dosage or cumulative (serial dosing), and can be readily determined by one skilled in the art. For instance, treatment, inhibition, reduction, prevention, mitigation, amelioration, or slowing of a viral ocular replication or infection, such as viral conjunctivitis, epidemic keratoconjunctivitis, and/or pharyngoconjunctival fever, reduction or suppression of a level of an inflammation inducing molecule and/or an inflammation inducing prostaglandin, stimulation or enhancement of a peroxisome proliferator-activated receptor (PPAR) pathway signal, promotion of the resolving phenotypic change of M1 to M2, modulation of Th1 and Th2 cytokines, and/or reduction or suppression of a NFκB pathway signal may comprise a one-time administration of an effective amount of a product combination disclosed herein. As a non-limiting example, an effective amount of product combination disclosed herein or a pharmaceutical composition disclosed herein can be administered once to an individual, e.g., as a single application. Alternatively, treatment, inhibition, reduction, prevention, mitigation, amelioration, or slowing of a viral ocular replication or infection, such as viral conjunctivitis, epidemic keratoconjunctivitis, and/or pharyngoconjunctival fever, reduction or suppression of a level of an inflammation inducing molecule and/or an inflammation inducing prostaglandin, stimulation or enhancement of a peroxisome proliferator-activated receptor (PPAR) pathway signal, promotion of the resolving phenotypic change of M1 to M2, modulation of Th1 and Th2 cytokines, and/or reduction or suppression of a NFκB pathway signal may comprise multiple administrations of an effective amount of the product combination disclosed herein carried out over a range of time periods, such as, e.g., one or more times daily, once every few days, weekly, monthly or yearly. As a non-limiting example, the product combination disclosed herein can be administered one, two, three, four, five or six times daily to an individual. The timing of administration can vary from subject to subject, depending upon such factors as the severity of a subject's symptoms. For example, an effective amount of the product combination disclosed herein can be administered to a subject three to six time daily for an indefinite period of time, or until the subject no longer requires therapy. A person of ordinary skill in the art will recognize that the condition of the subject can be monitored throughout the course of treatment and that the effective amount of the product combination disclosed herein that is administered can be adjusted accordingly.

### IV. Methods of Treatment

Some embodiments provided herein relate to methods of using a product combination comprising one or more RNase and one or more additional therapeutic agent as described herein for treating, preventing, inhibiting, reducing, mitigating, ameliorating, or slowing an ocular replication or infection, such as a viral infection of the eye.

In some embodiments, the method includes administering to a subject in need thereof a product combination comprising a therapeutic effective amount of one or more RNases, such as a ranpirnase, amphinase, or variant, derivative, analogue, or fragment thereof and a therapeutic effective amount of one or more additional therapeutic agents, such as one or more vasoconstrictor, one or more antibiotic, one or more immunomodulatory compound, or one or more steroid, or a combination thereof.

Some embodiments provided herein relate to methods of reducing or suppressing a level of a virus, viral titer, viral replication, protein synthesis and/or tRNA in a subject. In some embodiments, the methods include administering to a subject in need thereof a product combination as described herein, comprising a first pharmaceutical composition comprising a therapeutic effective amount of one or more RNase and a second pharmaceutical composition comprising a therapeutic effective amount of one or more additional therapeutic agent. Such administration reduces or suppresses a level of a virus, viral titer, viral replication, protein synthesis and/or tRNA. Also disclosed is a use of the product combination described herein for reducing or suppressing a level of a virus, viral titer, viral replication, protein synthesis and/or tRNA in a subject.

In some embodiments is provided a method or use of treating, inhibiting, preventing, delaying, mitigating, ameliorating, or slowing a viral conjunctivitis, epidemic keratoconjunctivitis, or pharyngoconjunctival fever caused by a virus infection from the Adenoviridae or Herpesviridae family, such as, for example, Human adenovirus B, a Human adenovirus D, a Human adenovirus E, HSV, VZV, EBV, HZV, or CMV, in a subject in need thereof by administering to the subject a product combination as disclosed herein, wherein administration reduces a symptom associated with the viral conjunctivitis. In some embodiments, the method disclosed herein treats a viral conjunctivitis, epidemic keratoconjunctivitis, or pharyngoconjunctival fever caused by human adenovirus B serotype 3, human adenovirus B serotype 7, human adenovirus B serotype 11, human adenovirus D serotype 8, human adenovirus D serotype 13, human adenovirus D serotype 19, human adenovirus D serotype 37, human adenovirus E serotype 4, or any combination thereof.

Some embodiments provided herein relate to methods of reducing or suppressing a level of an inflammation inducing molecule in a subject. In some embodiments, the methods include administering to a subject in need thereof a product combination as described herein, comprising a first pharmaceutical composition comprising a therapeutic effective amount of one or more RNase and a second pharmaceutical composition comprising a therapeutic effective amount of one or more additional therapeutic agent. Such administration reduces or suppresses a level of an inflammation inducing molecule. Also disclosed is a use of the product combination for reducing or suppressing a level of an inflammation inducing molecule. An inflammation inducing molecule disclosed herein includes a substance P, a calcitonin gene-related peptide, a glutamate, or a combination thereof.

Some embodiments provided herein relate to methods of reducing or suppressing a level of an inflammation inducing prostaglandin in a subject. In some embodiments, the methods include administering to a subject in need thereof a product combination as described herein, comprising a first pharmaceutical composition comprising a therapeutic effective amount of one or more RNase and a second pharmaceutical composition comprising a therapeutic effective amount of one or more additional therapeutic agent. Such administration reduces or suppresses a level of an inflammation inducing prostaglandin. Also disclosed is a use of the product combination for reducing or suppressing a level of an inflammation inducing prostaglandin. An inflammation inducing prostaglandin includes 15dPGJ2.

Some embodiments provided herein relate to methods of stimulating or enhancing a peroxisome proliferator-activated receptor (PPAR) signaling pathway activity in a subject. In some embodiments, the methods include administering to a subject in need thereof a product combination as described herein, comprising a first pharmaceutical composition comprising a therapeutic effective amount of one or more RNase and a second pharmaceutical composition comprising a therapeutic effective amount of one or more additional therapeutic agent. Such administration stimulates or enhances a PPAR signaling pathway activity. Also disclosed is a use of the product combination for stimulating or enhancing a PPAR signaling pathway activity. A PPAR signaling pathway activity includes a PPAR-α signaling pathway activity, a PPAR-γ signaling pathway activity, and a PPAR-δ (also known as PPAR-β) signaling pathway activity

Some embodiments provided herein relate to methods of promoting the resolving phenotypic change of M1 to M2 in a subject. In some embodiments, the methods include administering to a subject in need thereof a product combination as described herein, comprising a first pharmaceutical composition comprising a therapeutic effective amount of one or more RNase and a second pharmaceutical composition comprising a therapeutic effective amount of one or more additional therapeutic agent. Such administration induces apoptosis of Macrophage M1 cells, promotes differentiation of Macrophage M2 cells or both, thereby promoting the resolving phenotypic change of M1 to M2. Also disclosed is a use of the product combination for promoting the resolving phenotypic change of M1 to M2.

Some embodiments provided herein relate to methods of modulating a level of a Th1 cytokine and/or a level of a Th2 cytokine in a subject. In some embodiments, the methods include administering to a subject in need thereof a product combination as described herein, comprising a first pharmaceutical composition comprising a therapeutic effective amount of one or more RNase and a second pharmaceutical composition comprising a therapeutic effective amount of one or more additional therapeutic agent. Such administration reduces the levels of Interferon-gamma (IFNγ), Tumor necrosis factor-alpha (TNF-α), Interleukin-1b (IL-1b), Interleukin-12 (IL-12), or a combination thereof released from a Th1 cell, increases the level of IL-10 released from a Th2 cell, or both, thereby modulating a level of a Th1 cytokine and/or Th2 cytokine. Also disclosed is a use of the product combination for modulating a level of a Th1 cytokine and/or a level of a Th2 cytokine.

Some embodiments provided herein relate to methods of reducing or suppressing a NFκB signaling pathway activity in a subject. In some embodiments, the methods include administering to a subject in need thereof a product combination as described herein, comprising a first pharmaceutical composition comprising a therapeutic effective amount of one or more RNase and a second pharmaceutical composition comprising a therapeutic effective amount of one or more additional therapeutic agent. Such administration reduces or suppresses the NFκB signaling pathway activity. Also disclosed is a use of the product combination for reducing or suppressing a NFκB signaling pathway activity.

In some embodiments, the one or more RNase as described herein and the one or more additional therapeutic agent are combined in a product combination, as disclosed herein. In some embodiments, the product combination, including the combination of one or more RNase and one or more additional therapeutic agent together synergistically reduce, prevent, inhibit, mitigate, ameliorate, or treat an ocular replication or infection, such as a viral infection of the eye caused by a virus of the Adenoviridae or the Herpesviridae family. In some embodiments, the product combination reducing, inhibit, or suppress a level of virus, viral titer, viral replication, or viral functions, such as protein or RNA synthesis. In some embodiments, the product combination reduces, inhibits, or suppresses a level of virus, viral titer, or viral replication, or viral functions, such as protein or RNA synthesis by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, or in an amount within a range defined by any two of the aforementioned values. In some embodiments, the product combination reduces, inhibits, or suppresses a level of virus, viral titer, or viral replication, or viral functions, such as protein or RNA synthesis in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, or a range defined by any two of the aforementioned values.

In some embodiments, the product combination provided herein has an anti-inflammatory activity capable of reducing the levels of an inflammation inducing molecule. In some embodiments, the product combination disclosed herein has an anti-inflammatory activity capable of reducing the levels of substance P (SP), calcitonin gene-related peptide (CGRP), glutamate, or a combination thereof. In other aspects of this embodiment, the product combination disclosed herein has an anti-inflammatory activity capable of reducing the levels of SP, CGRP, glutamate, or a combination thereof released from a sensory neuron by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or in an amount within a range defined by any two of the aforementioned values. In yet other aspects of this embodiment, the product combination disclosed herein has an anti-inflammatory activity capable of reducing the levels of SP, CGRP, glutamate, or a combination thereof released from a sensory neuron in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, or a range defined by any two of the aforementioned values.

In some embodiments, the product combination disclosed herein has an anti-inflammatory activity capable of reducing the levels of an inflammation inducing prostaglandin. In some embodiments, the product combination disclosed herein has an anti-inflammatory activity capable of reducing the levels of an inflammation inducing prostaglandin released from a sensory neuron by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or in an amount within a range defined by any two of the aforementioned values. In some embodiments, the product combination disclosed herein has an anti-inflammatory activity capable of reducing the levels of an inflammation inducing prostaglandin released from a sensory neuron in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, or a range defined by any two of the aforementioned values.

In some embodiments, the product combination disclosed herein has an anti-inflammatory activity substantially similar to 15dPGJ2. In some embodiments, the product combination disclosed herein an anti-inflammatory activity that is, e.g., at least 5%, at least 15%, at least 25%, at least 50%, at least 55%, at least 60%, at least 65%, at least 70%, at least 75%, at least 80%, at least 85%, at least 90% or at least 95%, of the activity observed for 15dPGJ2, or an amount within a range defined by any two of the aforementioned values. In some embodiments, the product combination disclosed herein an anti-inflammatory activity that is in a range from, e.g., about 5% to about 100%, about 50% to about 100%, about 60% to about 100%, about 70% to about 100%, about 80% to about 100%, about 25% to about 90%, about 50% to about 90%, about 60% to about 90%, about 70% to about 90%, about 80% to about 90%, about 25% to about 80%, about 50% to about 80%, about 60% to about 80%, about 70% to about 80%, about 25% to about 70%, about 50% to about 70%, about 25% to about 60%, about 50% to about 60%, or about 25% to about 50% of the activity observed for 15dPGJ2, or a range defined by any two of the aforementioned values.

In some embodiments, the product combination disclosed herein has an anti-inflammatory activity capable of stimulating or enhancing activity from all PPAR signaling pathways. In some embodiments, the product combination disclosed herein has an anti-inflammatory activity capable of stimulating or enhancing activity of one or two of the PPAR signaling pathways. In some embodiments, the product combination disclosed herein has an anti-inflammatory activity capable of stimulating or enhancing a PPAR-α signaling pathway activity. In some embodiments, the product combination disclosed herein stimulates or enhances a PPAR-α signaling pathway activity by, e.g., at least 5%, at least 15%, at least 25%, at least 50%, at least 60%, at least 70%, at least 80%, or at least 90%, or an amount within a range defined by any two of the aforementioned values. In some embodiments, the product combination disclosed herein stimulates or enhances a PPAR-α signaling pathway activity in a range from, e.g., about 5% to about 100%, about 50% to about 100%, about 60% to about 100%, about 70% to about 100%, about 80% to about 100%, about 25% to about 90%, about 50% to about 90%, about 60% to about 90%, about 70% to about 90%, about 80% to about 90%, about 25% to about 80%, about 50% to about 80%, about 60% to about 80%, about 70% to about 80%, about 25% to about 70%, about 50% to about 70%, about 25% to about 60%, about 50% to about 60%, or about 25% to about 50%, or a range defined by any two of the aforementioned values.

In some embodiments, the product combination disclosed herein has an anti-inflammatory activity capable of stimulating or enhancing a PPAR-δ signaling pathway activity. In some embodiments, the product combination disclosed herein stimulates or enhances a PPAR-δ signaling pathway activity by, e.g., at least 5%, at least 15%, at least 25%, at least 50%, at least 60%, at least 70%, at least 80%, or at least 90%, or an amount within a range defined by any two of the aforementioned values. In some embodiments, the product combination disclosed herein stimulates or enhances a PPAR-δ signaling pathway activity in a range from, e.g., about 5% to about 100%, about 50% to about 100%, about 60% to about 100%, about 70% to about 100%, about 80% to about 100%, about 25% to about 90%, about 50% to about 90%, about 60% to about 90%, about 70% to about 90%, about 80% to about 90%, about 25% to about 80%, about 50% to about 80%, about 60% to about 80%, about 70% to about 80%, about 25% to about 70%, about 50% to about 70%, about 25% to about 60%, about 50% to about 60%, or about 25% to about 50%, or a range defined by any two of the aforementioned values.

In some embodiments, the product combination disclosed herein has an anti-inflammatory activity capable of stimulating or enhancing a PPARγ signaling pathway activity. In some embodiments, the product combination disclosed herein stimulates or enhances a PPARγ signaling pathway activity by, e.g., at least 5%, at least 15%, at least 25%, at least 50%, at least 60%, at least 70%, at least 80%, or at least 90%, or an amount within a range defined by any two of the aforementioned values. In some embodiments, the product combination disclosed herein stimulates or enhances a PPARγ signaling pathway activity in a range from, e.g., about 5% to about 100%, about 50% to about 100%, about 60% to about 100%, about 70% to about 100%, about 80% to about 100%, about 25% to about 90%, about 50% to about 90%, about 60% to about 90%, about 70% to about 90%, about 80% to about 90%, about 25% to about 80%, about 50% to about 80%, about 60% to about 80%, about 70% to about 80%, about 25% to about 70%, about 50% to about 70%, about 25% to about 60%, about 50% to about 60%, or about 25% to about 50%, or a range defined by any two of the aforementioned values.

In some embodiments, the product combination disclosed herein has an anti-inflammatory activity capable of promoting the resolving phenotypic change of M1 to M2. In some embodiments, the product combination disclosed herein has an anti-inflammatory activity capable of inducing apoptosis of Macrophage M1 cells. In some embodiments, the product combination disclosed herein has an anti-inflammatory activity capable of promoting differentiation of Macrophage M2 cells. In some embodiments, the product combination disclosed herein has an anti-inflammatory activity capable of inducing apoptosis of Macrophage M1 cells and promoting differentiation of Macrophage M2 cells. In some embodiments, the product combination disclosed herein has an anti-inflammatory activity capable of modulating the levels of a Th1 cytokine and/or Th2 cytokine. In some embodiments, the product combination disclosed herein has an anti-inflammatory activity capable of reducing a level of Interferon-gamma (IFNγ), Tumor necrosis factor-alpha (TNF-α), Interleukin-1b (IL-1b), Interleukin-12 (IL-12), or a combination thereof released from a Th1 cell. In some embodiments, the product combination disclosed herein has an anti-inflammatory activity capable of reducing a level of IFNγ, TNF-α, IL-1b, IL-12, or a combination thereof released from a Th1 cell by, e.g., at least 10%, at least 20%, at least 30%, at least 40%, at least 50%, at least 60%, at least 70%, at least 80%, or at least 90%, or an amount within a range defined by any two of the aforementioned values. In some embodiments, the product combination disclosed herein has an anti-inflammatory activity capable of reducing a level of IFNγ, TNF-α, IL-1b, IL-12, or a combination thereof released from a Th1 cell in a range from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, or a range defined by any two of the aforementioned values.

In some embodiments, the product combination disclosed herein has an anti-inflammatory activity capable of increasing a level of IL-10 released from a Th2 cell. In some embodiments, the product combination disclosed herein has an anti-inflammatory activity capable of increasing a level of IL-10 released from a Th2 cell by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or an amount within a range defined by any two of the aforementioned values. In some embodiments, the product combination disclosed herein has an anti-inflammatory activity capable of increasing a level of IL-10 released from a Th2 cell in a range from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, or a range defined by any two of the aforementioned values.

In some embodiments, the product combination disclosed herein has an anti-inflammatory activity capable of reducing a level of IFNγ, TNF-α, IL-1b, IL-12, or a combination thereof released from a Th1 cell and increasing a level of IL-10 released from a Th2 cell. In some embodiments, the product combination disclosed herein has an anti-inflammatory activity capable of reducing a level of IFNγ, TNF-α, IL-1b, IL-12, or a combination thereof released from a Th1 cell by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or in an amount within a range defined by any two of the aforementioned values, and capable of increasing a level of IL-10 released from a Th2 cell by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or an amount within a range defined by any two of the aforementioned values. In some embodiments, the product combination disclosed herein has an anti-inflammatory activity capable of reducing a level of IFNγ, TNF-α, IL-1b, IL-12, or a combination thereof released from a Th1 cell in a range from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, or a range defined by any two of the aforementioned values, and capable of increasing a level of IL-10 released from a Th2 cell in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, or a range defined by any two of the aforementioned values.

In some embodiments, the product combination disclosed herein has an anti-inflammatory activity capable of reducing of suppressing a NFκB signaling pathway activity. In some embodiments, the product combination disclosed herein has an anti-inflammatory activity capable of reducing of suppressing a NFκB signaling pathway activity by, e.g., at least 10%, at least 20%, at least 30%, at least 40%, at least 50%, at least 60%, at least 70%, at least 80%, or at least 90%, or in an amount within a range defined by any two of the aforementioned values. In some embodiments, the product combination disclosed herein has an anti-inflammatory activity capable of reducing of suppressing a NFκB signaling pathway activity in a range from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, or a range defined by any two of the aforementioned values.

In some embodiments, the product combination disclosed herein is administered using an ophthalmic formulation and an ophthalmic route of delivery. For example, the product combination can be formulated as a topical formulation, such as, e.g., an eye drop, a punctal plug, a salve, an ointment, a lotion, as an enteral formulation, such as, e.g., a tablet, capsule, syrup, or as a parenteral formulation, such as, e.g., an injectable or an intraocular plug. Such formulations can be administered, e.g., ophthalmically via ocular instillation, ocular irrigation or topical implant (punctal plug) or parenterally via intraocular injection or implant, intravitreal injection, intracorneal injection or implant or subconjunctival injection or implant. In some embodiments, the one or more RNase is formulated as a separate formulation than the one or more additional therapeutic agents. In such embodiments, the separate formulations may be administered in combination. In some embodiments, the one or more RNase is formulated with the one or more additional therapeutic agents in the product combination as the same formulation.

In some embodiments is provided a method of treating, reducing, inhibiting, preventing, mitigating, ameliorating, or slowing a viral replication or infection, including symptoms of a viral infection, as described herein. In some embodiments, the methods including administering a product combination that includes one or more RNase as described herein and one or more additional therapeutic agents as described herein. As used herein, the term “administering” refers to any delivery mechanism that provides a product combination comprising one or more RNase and one or more additional therapeutic agent disclosed herein to a subject that results in a clinically, therapeutically, or experimentally beneficial result. The actual delivery mechanism used to administer a composition disclosed herein to a subject can be determined by a person of ordinary skill in the art by taking into account factors, including, without limitation, the type of ocular infection, such as a viral conjunctivitis, the location of the ocular infection, such as a viral conjunctivitis, the cause of the ocular infection, such as a viral conjunctivitis, the severity of the ocular infection, such as a viral conjunctivitis, the degree of relief desired for ocular infection, such as a viral conjunctivitis, the duration of relief desired for ocular infection, such as a viral conjunctivitis, the level of virus, viral titer, viral replication, protein synthesis, or tRNA desired to be treated, inhibited, mitigated, ameliorated, prevented, reduced or suppressed, the particular signally pathway, inflammatory molecule, prostaglandin, and/or cytokine being modulated, the particular viral pathogen, the particular RNase and additional therapeutic agent used, the rate of excretion of the particular RNase and additional therapeutic used, the pharmacodynamics of the particular RNase and additional therapeutic used, the nature of any additional compounds to be included in the pharmaceutical composition, the particular route of administration, the particular characteristics, history and risk factors of the subject, such as, e.g., age, weight, general health and the like, or any combination thereof.

In some embodiments, administering the product combination disclosed herein includes administering to a surface of a conjunctiva of a subject, administering to a surface of an eye of a subject, or administering to a surface of a conjunctiva and/or an eye of a subject.

In some embodiments, administering the product combination disclosed herein includes administering an implant to a conjunctiva of a subject, administering an implant to an eye of a subject, or administering an implant to a conjunctiva and/or an eye of a subject.

In some embodiments, administering the product combination disclosed herein includes ocular instillation, ocular irrigation, intraocular injection, intracorneal injection, intravitreal injection or a subconjunctival injection

In some embodiments, the product combination disclosed herein is administered in an amount sufficient to treat an ocular replication or infection, such as viral conjunctivitis, epidemic keratoconjunctivitis, and/or pharyngoconjunctival fever, reduce a level of an inflammation inducing molecule and/or an inflammation inducing prostaglandin, stimulate or enhance a peroxisome proliferator-activated receptor (PPAR) pathway signal, promote the resolving phenotypic change of M1 to M2, modulate Th1 and Th2 cytokines, and/or reduce or suppress a NFκB pathway signal. In some embodiments, the amount of one or more RNase and one or more additional therapeutic agent in the product combination is administered in an amount sufficient to reduce one or more physiological conditions or symptom associated with an ocular infection, such as viral conjunctivitis, epidemic keratoconjunctivitis, and/or pharyngoconjunctival fever, reduce a level of an inflammation inducing molecule and/or an inflammation inducing prostaglandin, stimulate or enhance a peroxisome proliferator-activated receptor (PPAR) pathway signal, promote the resolving phenotypic change of M1 to M2, modulate Th1 and Th2 cytokines, and/or reduce or suppress a NFκB pathway signal. As used herein, the term “amount sufficient” includes “effective amount”, “effective dose”, “therapeutically effective amount” or “therapeutically effective dose” and refers to an amount of an RNase and/or an additional therapeutic agent disclosed herein to achieve the desired therapeutic effect and includes an amount sufficient to reduce one or more physiological conditions or symptom associated with an ocular infection, such as viral conjunctivitis, epidemic keratoconjunctivitis, and/or pharyngoconjunctival fever, an amount sufficient to reduce a level of an inflammation inducing molecule and/or an inflammation inducing prostaglandin, an amount sufficient to stimulate or enhance a peroxisome proliferator-activated receptor (PPAR) pathway signal, an amount sufficient to promote the resolving phenotypic change of M1 to M2, an amount sufficient to modulate Th1 and Th2 cytokines, and/or an amount sufficient to reduce or suppress a NFκB pathway signal.

The actual effective amount of an RNase and/or an additional therapeutic agent to be administered to a subject can be determined by a person of ordinary skill in the art by taking into account factors, including, without limitation, the type of ocular infection, such as viral conjunctivitis, the location of the ocular infection, such as viral conjunctivitis, the cause of the ocular infection, such as viral conjunctivitis, the severity of the ocular infection, such as viral conjunctivitis, the degree of relief desired for ocular infection, such as viral conjunctivitis, the duration of relief desired for ocular infection, such as viral conjunctivitis, the level of virus, viral titer, viral replication, protein synthesis, or tRNA desired to be reduced or suppressed, the particular signally pathway, inflammatory molecule, prostaglandin, and/or cytokine being modulated, the particular viral pathogen, the specific RNase and additional therapeutic agent used, the rate of excretion of the particular RNase and additional therapeutic agent used, the pharmacodynamics of the particular RNase and additional therapeutic agent used, the nature of any additional compounds to be included in the pharmaceutical composition, the particular route of administration, the particular characteristics, history and risk factors of the subject, such as, e.g., age, weight, general health and the like, or any combination thereof. Additionally, where repeated administration of the product combination is used, the actual therapeutically effective amount may further depend upon factors, including, without limitation, the frequency of administration, the half-life of the RNase and additional therapeutic agent used, or any combination thereof. It is known by a person of ordinary skill in the art that an effective amount of an RNase and an additional therapeutic agent disclosed herein or a pharmaceutical composition disclosed herein can be extrapolated from in vitro assays and in vivo administration studies using animal models prior to administration to humans. Wide variations in the necessary effective amount are to be expected in view of the differing efficiencies of the various routes of administration. For instance, ophthalmic administration generally would be expected to require higher dosage levels than by oral administration, and oral administration generally would be expected to require higher dosage levels than administration by intravenous or intravitreal injection. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors.

In some embodiments, an effective amount of one or more RNase disclosed herein or one or more additional therapeutic agent disclosed herein is separately generally in the range of about 0.001 mg/kg/day to about 100 mg/kg/day. In some embodiments, an effective amount of the one or more RNase or the one or more additional therapeutic agent disclosed herein may be, separately at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, at least 50 mg/kg/day, at least 60 mg/kg/day, at least 70 mg/kg/day, at least 80 mg/kg/day, at least 90 mg/kg/day, or at least 100 mg/kg/day or an amount within a range defined by any two of the aforementioned values.

In some embodiments, the product combination is provided in a dosage amount. In some embodiments, the dosage amount is formulated in an ophthalmic composition. In some embodiments, the ophthalmic composition includes both ranpirnase and the one or more additional therapeutic agent, such as oxymetazoline and/or brimonidine. In some embodiments, the ophthalmic composition is provided in two separate compositions, one of which includes ranpirnase, and one of which includes the one or more additional therapeutic agents, such as oxymetazoline and/or brimonidine. In some embodiments, the ophthalmic composition is administered to the subject one or more times each day, such as 1, 2, 3, 4, 5, 6, 7, 8, 9, or 10 times each day. In some embodiments, the administration is provided by instilling one or more drops in one eye or both eyes at each dosage. In some embodiments, the ophthalmic composition is administered at a given frequency each day (such as one or more times each day), for a number of drops per eye (such as one or more drops per eye), over a course of one or more days, such as 1, 2, 3, 4, 5, 6, 7, 8, 9, 10, 15, 20, 25, 30, 40, 50, 60 or more days, or for a length of time within a range defined by any two of the aforementioned values.

In some embodiments, a drop for administration includes a volume ranging from about 5 μL to about 50 μL, such as 5, 10, 15, 20, 25, 30, 35, 40, 45, or 50 μL, or a volume within a range defined by any two of the aforementioned values.

In some embodiments, a unit dosage includes a single drop administered in each eye, wherein each drop comprises ranpirnase present in an amount of about 0.03% w/v and oxymetazoline in an amount of about 0.01% to about 0.025% w/v. In some embodiments, a daily dosage includes a single drop administered in each eye four times daily, wherein each drop comprises ranpirnase present in an amount of about 0.03% w/v and oxymetazoline in an amount of about 0.01% to about 0.025% w/v.

In some embodiments, a unit dosage includes a single drop administered in each eye, wherein each drop comprises ranpirnase present in an amount of about 0.03% w/v and brimonidine in an amount of about 0.01% to about 0.025% w/v. In some embodiments, a daily dosage includes a single drop administered in each eye four times daily, wherein each drop comprises ranpirnase present in an amount of about 0.03% w/v and brimonidine in an amount of about 0.01% to about 0.025% w/v.

In some embodiments, the dosage amount includes ranpirnase administered at a dose of about 0.03% w/v and oxymetazoline administered at a dose of about 0.01% to 0.025% w/v administered in one or more drops, one or more times daily. In some embodiments, an ophthalmic composition is administered in each eye of the subject four times daily, and the ophthalmic composition comprises ranpirnase in an amount of about 0.03% w/v and oxymetazoline in an amount of about 0.01% to about 0.025% w/v.

In some embodiments, the dosage amount includes ranpirnase administered at a dose of about 0.03% w/v and brimonidine administered at a dose of about 0.01% to 0.025% w/v administered in one or more drops, one or more times daily. In some embodiments, an ophthalmic composition is administered in each eye of the subject four times daily, and the ophthalmic composition comprises ranpirnase in an amount of about 0.03% w/v and brimonidine in an amount of about 0.01% to about 0.025% w/v.

### EXAMPLES

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

### Formulations of Ranpirnase and Therapeutic Agents

The following example provides formulations of ribonucleases with a therapeutic agent in a product combination formulated for ophthalmic administration.

A ophthalmic formulations of ranpirnase and oxymetazoline were prepared. The formulations included 0.03% w/v (25 mM) ranpirnase in PBS at a pH of 7.4, oxymetazoline in an amount ranging from 0.01% to 0.025% w/v, and benzalkonium chloride (BAK).

### Example 2

### Compatibility of Ranpirnase and Therapeutic Agents

The following example demonstrates the compatibility of ribonucleases with a therapeutic agent in a product combination formulated for ophthalmic administration.

A formulation was prepared having 0.03% w/v ranpirnase together with oxymetazoline or brimonidine. The formulations were analyzed by chromatography to verify whether the therapeutic agent interferes with ranpirnase. Several chromatography methods were employed, including cation exchange and size exclusion chromatography, as well as benzalkonium chloride (BAK) analysis.

For cation exchange chromatography, analysis was performed with the following solutions: diluent (PBS pH 7.4), oxymetazoline HCl (0.5 mg/mL), placebo, and ranpirnase. Oxymetazoline HCl (0.5 mg/mL) solution was prepared in diluent. Both ranpirnase and its placebo were removed from the refrigerator, warmed to room temperature, and vialed into HPLC vials.

Ranpirnase exhibited a peak at RT=20.8 mins with a shoulder peak at RT=17.7 mins. For the oxymetazoline chromatogram, no oxymetazoline peak was observed, except for small peak after the diluent peak at 1.4 mins. Thus, oxymetazoline does not appear to present any interferences in the cation exchange method.

For size exclusion chromatography, analysis was performed on the following solutions: diluent (PBS pH 7.4), oxymetazoline HCl (0.5 mg/mL), placebo, and ranpirnase. Oxymetazoline HCl (0.5 mg/mL) solution was prepared in diluent. Both ranpirnase and its placebo were removed from the refrigerator, warmed to room temperature, and vialed into HPLC vials.

Ranpirnase exhibited a peak at RT=17.4 mins. For the oxymetazoline chromatogram, no Oxymetazoline peak was observed. Thus, oxymetazoline does not appear to present any interferences in the size exclusion method.

The benzalkonium chloride (BAK) analysis was performed on the following solutions: diluent (PBS pH 7.4), oxymetazoline HCl (0.05 mg/mL), placebo, and ranpirnase. Oxymetazoline HCl (0.5 mg/mL) solution (10×) was diluted to 0.05 mg/mL in diluent. Both ranpirnase and its placebo were removed from the refrigerator, warmed to room temperature, and vialed into HPLC vials.

Diluent in the method was 50:50 water:methanol, but oxymetazoline solution was prepared in PBS solution in order to determine whether oxymetazoline interferes with BAK peaks.

Typical RT of the BAK peaks are C12=6.3 mins, C14=8.0 mins, C16=9.4 mins. Ranpirnase chromatogram exhibited peaks at RT 6.8 mins, 8.5 mins, and 9.9 mins. Oxymetazoline exhibited a peak at RT 2.36 mins, but did not interfere with BAK peaks. Oxymetazoline did not present any interferences in the BAK method.

These analyses demonstrate the compatibility of formulating ranpirnase with oxymetazoline in an ophthalmic formulation.

### Example 3

### Efficacy and Safety of Product Combinations

This example demonstrates the efficacy and safety of product combinations of ranpirnase and oxymetazoline formulated in an ophthalmic formulation for treating patients having acute adenoviral conjunctivitis.

An ophthalmic formulation having ranpirnase in an amount of about 0.03% w/v and oxymetazoline in an amount of about 0.025% w/v is prepared in vehicle. Control formulations are prepared having ranpirnase in an amount of about 0.03% w/v in vehicle, or vehicle only.

The formulations are instilled in each eye four times a day (QID) for five days. Patients are randomized in a 2:1:1 ratio to receive one dose of the combination formulation, ranpirnase in vehicle, or vehicle alone. The study is performed in a double-masked blind study. Enrollment includes 352 total patients, with 176 receiving the combination formulation, 88 receiving ranpirnase in vehicle, and 88 receiving vehicle only.

Inclusion criteria for the study includes the following. Each patient must:

1. Be ≥18 years of age at Visit 1 (Day 1, Baseline) of either sex or any race.
2. Be willing and able to provide informed consent either written, or if the patient is not able to read, provide consent as stipulated by local laws and Human Research Ethics Committee (HREC) guidelines.
3. Be willing and able to follow all instructions and attend all study visits.
4. Have a clinical diagnosis of suspected acute adenoviral conjunctivitis in at least 1 eye and the presence of both of the following minimal clinical signs in that same eye:
Bulbar conjunctival redness: a minimum grade of ‘1’ on 0-3 scale
Watery ocular discharge: a minimum grade of ‘1’ on a 0-3 scale
   5. Patient reported presence of signs and symptoms consistent with adenoviral conjunctivitis ≤72 hours in same eye prior to Visit 1 (Note, if one eye has signs/symptoms for >72 hours the fellow eye may still qualify if it has signs/symptoms for ≤72 hours and all other inclusion/exclusion criteria are satisfied).
   6. Have a positive AdenoPlus® test at Visit 1 in the same eye that meets the minimum 1+ grade for bulbar conjunctival redness and watery ocular discharge.
   7. Be willing to avoid disallowed medications and treatments (see exclusions 12, 14 and 19) for the duration of the study.
   8. Be willing to discontinue contact lens wear for the duration of the study.
   9. Agree to submit to a pregnancy test at Visit 1 prior to enrollment and at Visit 4, or not be of childbearing potential.
   10. Agree to use an acceptable method of contraception for the duration of the study or not be of childbearing potential. Acceptable methods of birth control include: oral, transdermal, injectable, or implantable contraception, intrauterine device, abstinence, and surgical sterilization of partner. Female patients are not of childbearing potential if they have had a hysterectomy, bilateral oophorectomy, bilateral tubal ligation, or are post-menopausal by at least 12 months.
   11. Have a Best Spectacle Corrected Visual Acuity (BSCVA) of 0.60 logarithm of the minimum angle of resolution (logMAR) or better in each eye as measured using an Early Treatment of Diabetic Retinopathy Study (ETDRS) chart or ETDRS-equivalent chart for illiterate patients.

Exclusion criteria for the study includes the following. Each patient must not:

1. Have known sensitivity or poor tolerance to any component of the study medications or diagnostics.
2. Have a history of ocular surgical intervention or trauma within 12 weeks prior to Visit 1 or planned for the period of the study.
3. Have presence of any active ocular inflammation (e.g., uveitis, allergic conjunctivitis, ocular rosacea, or iritis), other than acute adenoviral conjunctivitis.
4. Have clinical signs or presence of an ocular infection other than acute adenoviral conjunctivitis (e.g., bacterial, fungal, or other ocular viral infection, such as herpes).
5. Have the presence of corneal sub-epithelial infiltrates in the study eye at baseline.
6. Have presence of a pseudomembrane in the study eye at baseline requiring an intervention that is not included as part of the study protocol procedures.
7. Have a history of recurrent corneal erosion syndrome, ulcerative keratitis, or dry eye, including meibomian gland dysfunction and other ocular surface diseases.
8. Have a presence of significant blepharitis, lid abnormality, significant inflammation of the lid margin, or ptosis.
9. Have lacrimal duct obstruction in either eye.
10. Have presence of any other clinically significant findings during the slit lamp exam that may interfere with study parameters or otherwise confound the data as determined by the investigator.
11. Have any clinically significant retinal or optic nerve findings (as observed in the non-dilated fundus exam) or prior diagnoses in either eye that may interfere with study parameters or otherwise confound the data as determined by the investigator).
12. Have used any topical ocular or systemic anti-viral or topical ocular or systemic corticosteroid within 7 days of enrollment and do not plan to start any topical ocular or systemic anti-viral during study duration. Inhaled, intranasal, and topical dermatologic steroids (except on the face) are allowed during the study.
13. Initiate or continue the use of warm or cold compresses for the duration of the trial.
14. Have used any topical ophthalmic solutions, including tear substitutes and diagnostics, within 2 hours of Visit 1 and be unable to discontinue all topical ophthalmic solutions (including diagnostics, except as required by this protocol and antibiotics) for the duration of the study. In addition, if the patient has used an artificial tear or other topical ophthalmic formulated in a hydrogel within the past 72 hours.
15. Be currently pregnant, nursing, or planning a pregnancy; or be a woman that has a positive pregnancy test.
16. Have any uncontrolled (not on a stable regimen for the past 30 days) systemic disease or debilitating disease (e.g., cardiovascular disease, hypertension, diabetes, or cystic fibrosis) or taking medications known to impact the ocular surface and/or tear film.
17. Have a planned overnight hospitalization during the period of the study.
18. Have any uncontrolled (not on a stable regimen for the past 30 days) autoimmune disease or taking medications known to impact the ocular surface and/or tear film.
19. Have prior (within 30 days of beginning study treatment) or anticipated concurrent use of an investigational drug or device.
20. Have a condition or a situation which, in the investigator's opinion, may put the patient at increased risk, confound study data, or interfere significantly with the patient's study participation.
21. Be unlikely to follow study instructions or to complete all required study visits or has a condition or situation that in the investigator's opinion, may put the patient at significant risk, may confound the study results, or may interfere significantly with the patient's participation in the study.

Efficacy endpoints include the following. Primary efficacy endpoints include: Clinical improvement from baseline of acute adenoviral conjunctivitis in the study eye at Visit 2, as measured by the sum of the severity of bulbar conjunctival redness (graded on a scale of 0-3 using a validated picture-based reference scale) and watery conjunctival discharge

Key secondary efficacy endpoints include: 1. Adenoviral eradication at Visit 2, assessed by cell culture immunofluorescence assay (CC-IFA); 2. Global clinical cure of acute adenoviral conjunctivitis in the study eye at Visit 2 as measured by the absence (score=0) of the sum of the scores using the referenced scales for watery conjunctival discharge and bulbar conjunctival redness at each visit; and 3. Presence/absence of sub-epithelial infiltrates at Visit 5.

Secondary efficacy endpoints include: 1. Adenoviral eradication at each follow-up visit (besides visit 2) assessed by cell culture immunofluorescence assay (CC-IFA); 2. Mean change from baseline in viral titer levels in the study eye at each follow-up visit assessed by quantitative polymerase chain reaction (qPCR); 3. Percentage of patients with viral titers less than the Lower Limit of Detection (LLOD) assessed by qPCR; 4. Percentage of patients with viral titers less than 100 copies/mL assessed by qPCR; 5. Clinical improvement of acute adenoviral conjunctivitis in the study eye at each visit (Visit 2, Visit 3, Visit 4, or Visit 5 independently) as measured by severity of bulbar conjunctival redness (graded on a scale of 0-3 using a validated picture-based reference scale); 6. Clinical improvement of acute adenoviral conjunctivitis in the study eye at each visit (Visit 2, Visit 3, Visit 4, or Visit 5 independently) as measured by severity of watery conjunctival discharge (graded on a scale of 0-3 using a validated reference scale); 7. Clinical cure of acute adenoviral conjunctivitis in the study eye at each visit (Visit 2, Visit 3, Visit 4, or Visit 5 independently) as measured by the absence (score=0) of bulbar conjunctival redness (graded on a scale of 0-3 using a validated picture-based reference scale); 8. Clinical cure of acute adenoviral conjunctivitis in the study eye at each visit (Visit 2, Visit 3, Visit 4, or Visit 5 independently) as measured by the absence (score=0) of watery conjunctival discharge (graded on a scale of 0-3 using a validated reference scale); 9. Global clinical cure of acute adenoviral conjunctivitis in the study eye at each visit (Visit 3, Visit 4, or Visit 5 independently) as measured by the absence (score=0) of the sum of the scores using the referenced scales for watery conjunctival discharge and bulbar conjunctival redness; 10. Expanded clinical cure of adenoviral conjunctivitis in the study eye at each visit (Visit 2, Visit 3, Visit 4, or Visit 5 independently). Expanded clinical cure is defined as a score of 0 or 1 for the following 2 clinical signs: watery conjunctival discharge and bulbar conjunctival redness; 11. Mean change from baseline in bulbar conjunctival redness score in the study eye at each visit; 12. Mean change from baseline in watery conjunctival discharge score in the study eye at each visit; 13. Mean change from baseline in the sum of scores for bulbar conjunctival redness and watery conjunctival discharge at each visit; 14. Presence/absence of sub-epithelial infiltrates at each visit other than Visit 5; 15. Severity of sub-epithelial infiltrates at each visit; and 16. Severity of other clinical signs and symptoms for each visit of the following: Tearing, Burning, Blurry vision, Photophobia, Foreign body sensation, Itching, Lid edema, or Lid erythema.

Safety measures are assessed by: 1. Slit Lamp Biomicroscopy (non-dilated fundus examination); 2. BSCVA; 3. Urine Pregnancy Testing; 4. Adverse Events (AEs); 5. Tolerability by assessment of drop comfort in the fellow eye at Visit 1 by the patient upon instillation (self-administered), at 5 minutes after instillation using visual analog scales as defined in the Study Reference Manual.

Summaries for continuous variables include the sample size, mean, standard deviation, median, minimum, and maximum. Summaries for discrete variables include frequencies and percentages. In general, data is summarized by treatment group. Differences between treatment groups are calculated as Test—Vehicle and change from baseline (CFB) are calculated as follow-up visit—baseline. The baseline visit is defined as the last non-missing measure prior to initiation of investigational treatment. All efficacy analyses are a two-sided alpha=0.05 test, unless otherwise stated.

Study populations include the following. The intent-to-treat (ITT) population consists of all randomized patients; Subjects in the ITT are analyzed under the treatment to which they were randomized.

The modified intent-to-treat (mITT) population consists of a subset of ITT patients who have received a least one (1) dose of investigational product and have a positive CCIFA at Visit 1 in an eye that meets the clinical symptom requirements (1+ grade for bulbar conjunctival redness and watery ocular discharge) at Visit 1. The mITT population are used for the efficacy analysis and analyze patients under the treatment to which they were randomized.

The per-protocol (PP) population is a subset of the mITT population and includes the patients who do not have major protocol violations likely to seriously affect the primary outcome of the study. The PP population is used for sensitivity analysis of efficacy, analyzing patients under the treatment actually received. Important protocol deviations related to study inclusion or exclusion criteria, conduct of the trial, patient management, or patient assessment identified prior to unmasking treatment.

The safety population includes all randomized patients who receive at least one dose of study medication. The safety population is analyzed as treated and is used for the safety analyses. No data is excluded for any reason.

The unit of analysis are the study eye for all efficacy and ocular safety summaries. If both eyes of a subject meet clinical symptom requirements (1+ grade for bulbar conjunctival redness and watery ocular discharge) at Visit 1 and are CC-IFA positive at Visit 1, then the study eye is the eye with the highest Visit 1 total clinical symptom score (bulbar conjunctival redness+watery ocular discharge). If both eyes have the same Visit 1 total clinical symptom score, then the study eye is the right eye (OD); the other eye is considered the qualified fellow eye. If only one eye of a subject meets the clinical symptom requirements (1+ grade for bulbar conjunctival redness and watery ocular discharge) at Visit 1 and are CC-IFA positive at Visit 1, then that eye is the study eye. The other eye is considered a non-qualified fellow eye.

The study eye for the ITT and Safety populations is defined as the study eye for the mITT population, for those subjects in the mITT population. For those subjects not in the mITT population, the study eye is defined as the eye with the highest Visit 1 total clinical symptom score (bulbar conjunctival redness+watery ocular discharge). If both eyes have the same Visit 1 total clinical symptom score, then the study eye is the right eye (OD). If the other eye meets the clinical symptom score requirements (1+ grade for bulbar conjunctival redness and watery ocular discharge) at Visit 1 then the other eye is considered a qualified fellow eye. Otherwise, the other eye is considered a non-qualified fellow eye. Subject level measures are presented at the subject level.

Subjects treated with the formulation exhibit improvements in eye infections. The difference between study eyes treated with the combination formation (ranpirnase and oxymetazoline) and study eyes treated with vehicle alone, in the mean change from baseline (CFB) total clinical sign score (bulbar conjunctival redness+watery ocular discharge) at Visit 2≠0, such that the combination formulation is superior to the vehicle alone in mean CFB total clinical sign score at Visit 2, and the following key secondary endpoints are tested in the following hierarchical order at a two-sided alpha=0.05:

The difference, between study eyes treated with the combination formulation and study eyes treated with vehicle, in the percentage of study eyes with adenoviral eradication (assessed by CC-IFA) at Visit 2=0.

The difference, between study eyes treated with the combination formulation and study eyes treated with vehicle, in the percentage of study eyes with adenoviral eradication (assessed by CC-IFA) at Visit 2≠0.

The difference, between study eyes treated with the combination formulation and study eyes treated with ranpirnase in vehicle, in the mean change from baseline (CFB) total clinical sign score (bulbar conjunctival redness+watery ocular discharge) at Visit 2=0.

The difference, between study eyes treated with the combination formulation and study eyes treated with ranpirnase in vehicle, in the mean CFB total clinical sign score (bulbar conjunctival redness+watery ocular discharge) at Visit 2≠0.

The difference, between study eyes treated with ranpirnase in vehicle and study eyes treated with vehicle, in the percentage of study eyes with adenoviral eradication (assessed by CC-IFA) at Visit 2=0.

The difference, between study eyes treated with ranpirnase in vehicle and study eyes treated with vehicle, in the percentage of study eyes with adenoviral eradication (assessed by CC-IFA) at Visit 2≠0.

The difference, between study eyes treated with the combination formulation and study eyes treated with vehicle, in the percentage of study eyes with global clinical cure at Visit 2=0.

The difference, between study eyes treated with the combination formulation and study eyes treated with vehicle, in the percentage of study eyes with global clinical cure at Visit 2≠0.

The difference, between study eyes treated with the combination formulation and study eyes treated with vehicle, in the percentage of study eyes with absence of sub-epithelial infiltrates at Visit 5=0.

The difference, between study eyes treated with the combination formulation and study eyes treated with vehicle, in the percentage of study eyes with absence of sub-epithelial infiltrates at Visit 5≠0.

Hierarchical fixed sequence testing is employed to maintain the type I error rate.

An interim analysis is performed after approximately 50% of the patients have been randomized and either completed through Visit 2 or discontinued the study. The decision to be made at the interim analysis is whether to stop the trial for futility based on the primary endpoint: mean CFB total clinical symptom score at Visit 2. Group sequential methods are used with an O'Brien-Fleming alpha-spending function for superiority (Zstatistic for superiority at the interim=2.96259, corresponding to a one-sided alpha=0.00153; note, this is an Type I Error penalty for an interim analysis, there is no intent on stopping the study at the interim for superior efficacy) and a non-binding Power Family alpha-spending function (with Phi parameter=3) for futility (Zstatistic for futility at the interim=0.02379, corresponding to a one-sided alpha=0.49055). With this design the one-sided alpha remaining for the final analysis is 0.02450 (corresponding to a two-sided alpha=0.049 and a Zstatistic=1.96860).

With an 80% culture-confirmation rate from a positive AdenoPlus® test, approximately 352 patients are randomized in this study. A sample size of 98 mITT subjects (study eyes) in the treatment group and 49 mITT subjects (study eyes) in the vehicle treatment group yields 90% power to detect a difference in study eye mean CFB total clinical symptom score at Visit 2 assuming a true difference in study eye mean CFB total clinical symptom score at Visit 2 of −1.0, a common standard deviation of 1.75, and the specified group sequential interim analysis strategy. Assuming a sample size of 140 mITT subjects (study eyes) in the treatment group and 70 mITT subjects (study eyes) in the vehicle treatment group toward developing the safety profile of the combination formulation, the study has >97% power for the primary endpoint.

Additionally, 140 mITT subjects (study eyes) in the treatment group and 70 mITT subjects (study eyes) in the vehicle treatment group yields >97% power to detect a difference in the proportion of study eyes with adenoviral eradication by CC-IFA assay at Visit 2, assuming the true proportion of study eyes with adenoviral eradication by CC-IFA assay is 0.65 for the combination formulation and 0.35 for vehicle and a 2-sided alpha=0.049.

Seventy (70) mITT subjects (study eyes) in the ranpirnase vehicle treatment group and 70 mITT subjects (study eyes) in the vehicle treatment group yields 95% power to detect a difference in the proportion of study eyes with adenoviral eradication by CC-IFA assay at Visit 2, assuming the true proportion of study eyes with adenoviral eradication by CC-IFA assay is 0.65 for the combination formulation and 0.35 for vehicle and a 2-sided alpha=0.049.

The planned sample size yields >95% power to demonstrate superiority of the combination formulation to vehicle in both the study eye mean CFB total clinical symptom score at Visit 2 and the proportion of study eyes with adenoviral eradication by CC-IFA assay at Visit 2.

The primary analysis of the primary and key secondary efficacy endpoints are conducted in the mITT population and utilize observed data only unless the >5% of primary efficacy measures are missing in which case intercurrent events handled in the following manners:

1) Discontinuation of study drug and non-optimal compliance is ignored [treatment policy strategy].

2) Withdrawal due to lack of efficacy or adverse events, missing data is: a. Multiply imputed for study eye CFB total clinical symptom score using vehicle-based Markov Chain Monte Carlo (MCMC) methodology for nonmonotone missing and regression methodology for monotone missing [hypothetical strategy]; b. Singly imputed as failure for study eye adenoviral eradication; c. Singly imputed as failure for global clinical cure of acute adenoviral conjunctivitis in the study eye [hypothetical strategy]; d. Multiply imputed for study eye sub-epithelial infiltrates using vehicle-based Markov Chain Monte Carlo (MCMC) methodology for nonmonotone missing and logistic regression methodology for monotone missing [hypothetical strategy].

3) Missing data without withdrawal or withdrawal due to reasons other than lack of efficacy or adverse events, missing data is: a. Multiply imputed for study eye CFB total clinical symptom score using treatment-based Markov Chain Monte Carlo (MCMC) methodology for nonmonotone missing and regression methodology for monotone missing [hypothetical strategy]; b. Multiply imputed for study eye adenoviral levels (with adenoviral eradication determined therefrom) using treatment-based Markov Chain Monte Carlo (MCMC) methodology for nonmonotone missing and regression methodology for monotone missing [hypothetical strategy]; c. Multiply imputed for study eye CFB total clinical symptom score (with global clinical cure of acute adenoviral conjunctivitis determined therefrom) using treatment-based Markov Chain Monte Carlo (MCMC) methodology for nonmonotone missing and regression methodology for monotone missing [hypothetical strategy]; d. Multiply imputed for study eye sub-epithelial infiltrates using treatment-based Markov Chain Monte Carlo (MCMC) methodology for nonmonotone missing and logistic regression methodology for monotone missing [hypothetical strategy]. Multiple imputations of missing values for the dichotomous key secondary endpoints of adenoviral eradication (assessed by CC-IFA) and global clinical cure of acute adenoviral conjunctivitis are completed for the continuous measure (adenoviral levels assessed by CC-IFA and CFB total clinical symptom score, respectively), then the response variables are determined therefrom.

Sensitivity analyses on the primary and key secondary efficacy variables are performed using the mITT with all missing data imputed as failure (adenoviral eradication, global clinical cure, and sub-epithelial infiltrates) and multiply imputed using vehicle-based methodology (CFB total clinical symptom score); the mITT with all missing data imputed as success (adenoviral eradication, global clinical cure, and sub-epithelial infiltrates) and multiply imputed using treatment-based methodology (CFB total clinical symptom score); mITT with observed data, and PP set with observed data. Additional sensitivity analyses such as tipping point may be performed and are specified in the Statistical Analysis Plan (SAP).

The primary efficacy endpoint of CFB total clinical symptom score at Visit 2 is summarized using continuous summary statistics by treatment group. The primary analysis of the primary efficacy endpoint of CFB total clinical symptom score is completed using a linear model including fixed effects of baseline total clinical symptom score as a covariate and treatment. The least squares mean (LSM) CFB total clinical symptom score and the difference in LSM CFB total clinical symptom score along with corresponding two-sided 95% Confidence Intervals (CIs) and p-values are presented. Treatment comparisons are made using two-sample t-tests as a sensitivity analysis to the primary model above.

The key secondary efficacy endpoints of adenoviral eradication (assessed by CC-IFA) at Visit 2, CFB total clinical symptom score at Visit 2, adenoviral eradication (assessed by CC-IFA), global clinical cure at Visit 2, and absence of sub-epithelial infiltrates at Visit 5 are summarized using discrete or continuous (CFB total clinical symptom score only) summary statistics by treatment group. The primary analysis of each of the key secondary efficacy endpoints of adenoviral eradication and global clinical cure is completed using a logistic regression model including fixed effects of corresponding baseline score as a covariate and treatment. The adjusted odds ratios and marginal proportions of adenoviral eradication (and clinical cure) and difference in marginal proportions of adenoviral eradication (and clinical cure) along with corresponding two-sided 95% Confidence Intervals (CIs) and p-values are presented.

Treatment comparisons are made using Pearson's chi-squared test or Fisher's exact test (if any of the expected cell counts are less than five) as a sensitivity analysis to the primary model above and as the primary analysis of sub-epithelial infiltrates. The primary analysis of the key secondary efficacy endpoint of CFB total clinical symptom score at Visit 2 between the combination formulation and the ranpirnase in vehicle is completed using the same analysis strategy as the primary analysis of the primary efficacy endpoint.

Change from baseline total clinical symptom score is summarized using continuous summary statistics for the ITT population and is tested between treatment groups using the same strategy as for the mITT population, using the primary imputation strategy as well as using observed data only.

The safety analysis summarizes treatment-emergent AEs (TEAEs), including both ocular and systemic TEAEs, in the study eye and fellow eye for all treated patients using discrete summaries at the patient and event levels. All TEAEs (defined as an AE that occurs or worsens on or after the first treatment) are coded using Medical Dictionary for Regulatory Activities to identify system organ class and preferred term.

Slit lamp biomicroscopy measures are summarized at each visit using discrete summary statistics. BSCVA data are summarized at each visit, using discrete summaries, including change from baseline in the number of lines and the proportion of patients with change from previous visit of ≥3 lines (>0.3 LogMAR).

The demographic characteristics (i.e., age, sex, race, ethnicity, and iris color), medical history, and ocular history data are summarized and presented by treatment group and as an overall summary of all patients using discrete or continuous summary statistics as appropriate.

### Example 4

### In Vivo Antiviral Effects of the Product Combination

The antiviral activity of the product combination is evaluated in vivo using an ocular rabbit replication model. To conduct this assay, 25 New Zealand white (NZW) rabbits are anesthetized using the general anesthesia ketamine and xylazine and the topical anesthesia proparacaine. Each rabbit is topically inoculated with 50 μL of Adenovirus serotype Ad5 (3×10⁷ pfu/mL) in both eyes following corneal epithelial scarification (12 cross-hatched strokes of a 25 sterile needle). Eyes are closed and gently rubbed for 5 seconds to ensure contact of the virus on all ocular surfaces. Inoculation of both eyes allows for the reduction in the number of animals needed without jeopardizing statistical validity. Twenty-four hours later, rabbits are randomly assigned to one of five topical treatment groups: (1A) 25 μM ranpirnase alone (n=5); (1B) 25 μM ranpirnase+one or more additional therapeutic agent (n=5); (1C) 10 μM ranpirnase+one or more additional therapeutic agent (n=5); (1D) 0.9% saline, as the negative control (n=5); and (CC) 0.5% cidofovir, as the positive control (n=5). Treatment rabbits (1A, 1B, 1C, and 1D) are treated in both eyes eight times daily for 9 days. The control group (CC) are treated in both eyes twice daily for 7 days. All topical solutions (37 μL drops) are instilled with an electronic pipette (EDP; Rainin, Oakland, Calif.) set in the multi-dispense mode. Ocular swabbing is performed to recover adenovirus from tear film and corneal and conjunctival surfaces, after topical anesthesia with proparacaine, at least 1 hour after the final dose on days 0, 1, 3, 4, 5, 7, 9, 11, and 14 after inoculation. The ocular samples from each eye are placed individually into tubes containing 1 mL of medium and are frozen at −70° C. pending viral plaque assay.

The ocular samples are assayed for Ad5 titers by performing a plaque reduction assay. Samples are diluted 1:10 and these dilutions are inoculated onto duplicate wells of a 24 well multi-plate containing A549 monolayers. The virus is adsorbed for 3 hours at 37° C. in a 5% CO₂-water vapor atmosphere. After adsorption, 1 mL of medium plus 0.5% methylcellulose is added to each well, and the plates are incubated at 37° C. in a 5% CO₂-water vapor atmosphere. After 7 days, the cells are stained with 0.5% gentian violet, and the number of plaques is counted using a dissecting microscope (25×). The viral titers are then calculated and are expressed as plaque-forming units per milliliter (PFU/mL). Data from the study are analyzed using analysis of variance (ANOVA) with Fisher's pair-wise comparisons and X2 analyses using True Epistat and/or Minitab statistical software. Significance is established at the P≤0.05 confidence level.

Animals receiving the product combination exhibit reduction in viral replication or infection of the eye as compared to animals that received ranpirnase alone (1A), saline alone (1D), or cidofovir alone (CC).

### Example 5

### In Vivo Anti-Herpetic Effects of the Product Combination

The antiviral efficacy of the product combination is evaluated in vivo using a herpetic rabbit model for evaluating both viral replication and clinical signs and symptoms. To conduct this assay, 20 female rabbits weighing 1.5 to 2.0 kilograms are anesthetized using the general anesthesia ketamine (40 mg/kg) and xylazine (4 mg/kg) and the topical anesthesia proparacaine.

On day 1, each rabbit is topically inoculated with 50 μL of HSV-1 (3.2×10⁵ pfu/eye) in both eyes following corneal epithelial scarification (3 interlocking circles of a 7.5 mm trephine). Eyes are closed and gently rubbed for 5 seconds to ensure contact of the virus on all ocular surfaces.

Rabbits are randomly assigned to one of four topical treatment groups: (1) negative control including pharmaceutical carrier only; (2) test composition of 10 μM ranpirnase+one or more additional therapeutic agent; (3) test composition of 25 μM ranpirnase+one or more additional therapeutic agent; and (4) positive control of 0.15% ganciclovir ophthalmic gel. Each group includes 5 rabbits. Rabbits are treated in both eyes four times daily for 10 days for each of groups 1, 2, and 3, and five times daily for 10 days for group 4. Treatment is initiated on day 2.

On each of days 2, 3, 5, 7, 9, 11, and 14, both eyes of each rabbit are examined using slit-lamp examination to grade HSV-1 dendritic keratitis on a scale of 0 to 4. Slit-lamp examination is carried out with 0.1% sodium fluorescein and cobalt blue filter to visualize the typical corneal epithelial keratitis produced by an ocular HSV-1 infection, and HSV-1 dendritic keratitis is evaluated and graded at each examination. The dendritic keratitis grade scale is as follows:
0=No Dendrites
0.5=1-5 Dendrites
1.0=6-10 Dendrites
1.5=11-15 Dendrites
2.0=16-20 Dendrites or Geographic Ulcer <¼ of the Corneal Surface
2.5=>20 Dendrites or Geographic Ulcer >¼ but <⅓ of the Corneal Surface
3.0=Geographic Ulcer or Multiple Dendrites >¼ but <½ of the Corneal Surface with Stromal Involvement
3.5=Geographic Ulcer or Multiple Dendrites >½ but <⅔ of the Corneal Surface with Stromal Involvement
4.0=Geographic Ulcer or Multiple Dendrites >⅔ of the Corneal Surface with Stromal Involvement

Ocular viral cultures are also obtained after each slit-lamp examination by swabbing each eye in the upper and lower fornices with a cotton-tipped applicator following topical anesthesia with 0.5% proparacaine. Ocular viral cultures are obtained at least 1 hour after the final dose of test drug. The corneas are not cultured to avoid spreading the lesions. The swabs from each eye are placed individually into tubes containing 1 ml of outgrowth media, and frozen at −80° C. pending viral plaque assay.

Each frozen HSV-1 ocular sample to be titered are thawed and diluted serially (1:10) for three dilutions. Each dilution (0.1 ml per well) is then inoculated onto Vero or A549 cells in duplicate wells of a 24 well plate. The virus is adsorbed for 1 hour at 37° C. in a 5% CO₂-water vapor atmosphere. Following adsorption, 1 ml of outgrowth media plus 0.5% methylcellulose is added to each well, and the plates are incubated at 37° C. in a 5% CO₂-water vapor atmosphere. The plates are stained with 0.5% gentian violet after 5 days, and the number of plaques per well are counted under a dissecting microscope (25×). The ocular HSV-1 titers are calculated and expressed as plaque-forming units per ml (PFU/ml). The data is analyzed statistically using True Epistat and/or Minitab statistical software. Outcome measures include Daily HSV-1-Positive Cultures per Total Cultures, Daily Viral Titers, Duration of Shedding, Daily Keratitis Scores, Number of Eyes with Keratitis, and Time to Resolution of Keratitis. Significance is established at the p≤0.05 confidence level.

Animals receiving the product combination exhibit reduction in viral replication or infection of the eye and improved clinical signs/symptoms based on the dendritic keratitis grade scale, as compared to animals that received carrier alone (1) or ganciclovir alone (4).

### Example 6

### In Vivo Anti-Hyperemia Effects of the Product Combination

The anti-hyperemia activity of the product combination is evaluated in vivo using rabbit model of hyperemia. To conduct this assay, 25 New Zealand white (NZW) rabbits are anesthetized using the general anesthesia ketamine and xylazine and the topical anesthesia proparacaine. Hyperemia is induced in both eyes of each rabbit by topically administering 0.01% histamine or 0.3% arachidonic acid. The degree or severity of hyperemia is assessed in each rabbit.

Twenty-four hours later, rabbits are randomly assigned to one of five topical treatment groups: (1A) 25 μM ranpirnase alone (n=5); (1B) 25 μM ranpirnase+one or more additional therapeutic agent, such as a vasoconstrictor (n=5); (1C) 10 μM ranpirnase+one or more additional therapeutic agent, such as a vasoconstrictor (n=5); (1D) 0.9% saline, as the negative control (n=5); and (CN) 0.1% naphazoline, as the positive control (n=5). All rabbit groups (1A, 1B, 1C, 1D, and CN) are treated in both eyes eight times daily for 9 days. All topical solutions (37 μL drops) are instilled with an electronic pipette (EDP; Rainin, Oakland, Calif.) set in the multi-dispense mode. The degree of hyperemia is monitored daily during the course of treatment.

Animals receiving the product combination exhibit reduction in hyperemia of the eye as compared to animals that received ranpirnase alone (1A), saline alone (1D), or naphazoline alone (CDV).

### Example 7

### Arterial Explant Culture Models of Vasoconstriction

Explant cultures of artery is provided in culture as a model of vasoconstriction. Explant cultures are prepared by obtaining arteries. Arterial segments are transferred to chambered coverslips containing culture medium. Explant cultures are treated with the treatment groups described in Example 6, namely: (1A) 25 μM ranpirnase alone; (1B) 25 μM ranpirnase+one or more additional therapeutic agent, such as a vasoconstrictor; (1C) 10 μM ranpirnase+one or more additional therapeutic agent, such as a vasoconstrictor; and (CN) 0.1% naphazoline, as the positive control. A negative control of culture medium alone is used. Culture medium and treatment agents are replaced daily.

Arterial explant cultures are monitored for effects of vasoconstriction in each group. Cultures receiving the product combination exhibit improved effects of vasoconstriction compared to cultures receiving ranpirnase alone, naphazoline alone, or culture medium alone.

### Example 8

### Ocular Tolerability and Toxicity of Product Combination

The tolerability and toxicity of the product combination is assessed.

To assess tolerability of the product combination, the NZW rabbits as treated in Examples 4 and 5 are assayed for eye irritation using a Draize scale for ocular lesions. Classification of eye irritation is evaluated for both eyes of each rabbit on day 3 and day 9 using the maximum mean total score (MMTS). The MMTS score is as follows: 0.0-0.5, Non-Irritating (N); 0.6-2.5, Practically Non-Irritating (PN); 2.6-15.0, Minimally Irritating (M1); 15.1-25.0, Mildly Irritating (M2); 25.1-50.0, Moderately Irritating (M3); 50.1-80.0, Severely Irritating (S); 80.1-100.0, Extremely Irritating (E); and 100.1-110.0, Maximally Irritating (Mx). The product combination is well tolerated, with low irritation.

To assess in vitro cytotoxicity, 96-well plates are seeded with A549 cells at 1×10⁵ cells/mL and incubated overnight at 37° C. with 5% CO₂. The product combination is serially diluted to include ranpirnase in concentrations of 1.0 μM, 10 μM and 50 μM. After removal of the tissue culture media, 100 μL of each dilution is added to 3 wells of a 96-well plate with 80% to 100% confluent cells. As controls, 100 μL of a lysis buffer containing 0.25% TRITON X-100 is added to 6 wells (positive cytotoxicity control) and 100 μL of tissue culture media with no ranpirnase or ranpirnase alone is added to 6 wells (negative cytotoxicity control). Each test and control treatment is incubated on the A549 monolayers for 2 days at 37° C. with 5% CO₂. A 100 μL aliquot of the fluorometric stain is added to each well, and the cells are incubated for 1 hour at 37° C. with 5% CO₂. The fluorometric stain (ALAMARBLUE®, Invitrogen, Carlsbad, Calif.) acts as a redox indicator that is reduced to a fluorescent form by metabolically active living cells. Fluorescence is read with a plate reader (Biotek Synergy 2; Biotek), with a 500/27-nm excitation filter and a 620/40-nm emission filter, at a sensitivity of 35. Cytotoxicity is determined by the percentage of residual viable cells after exposure to Ranpirnase (% cytotoxicity=100−[(median florescence drug/median fluorescence no drug)×100], where “drug” is either one of the three concentrations of ranpirnase in the product combination or the lysis buffer and “no drug” or “ranpirnase alone” is the negative control. The observed differences are evaluated statistically using the non-parametric Kruskal-Wallis ANOVA and Duncan's Multiple Comparisons and significance is established at the P≤0.05 confidence level.

The experiments reveal that the product combination comprising ranpirnase produced significant cytotoxicity in A549 cells after 2 days of exposure.

It is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions, and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies may be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The terminology used herein is for the purpose of describing particular embodiments only and is not intended to limit the scope of the present invention, which is defined solely by the claims. Accordingly, the present invention is not limited to that precisely as shown and described.

What is claimed is:

1. A product combination that inhibits or slows an ocular infection, wherein the product combination comprises:
a therapeutically effective amount of one or more ribonuclease (RNase); and
a therapeutically effective amount of one or more additional therapeutic agent, wherein the additional therapeutic agent is a vasoconstrictor, an antibiotic, an immunomodulatory compound, a steroid, or a combination thereof.

2. The product combination of claim 1, wherein the one or more RNase is ranpirnase, an analogue, variant, derivative, or fragment thereof.

3. The product combination of claim 2, wherein the one or more ranpirnase, analogue, variant, derivative, or fragment thereof is present in an amount of about 0.001% to about 1% w/v.

4. The product combination of any one of claims 1-3, wherein the one or more additional therapeutic agent is naphazoline, tetrahydrozoline, phenylephrine, oxymetazoline, brimonidine, apraclonidine, ephedrine, azithromycin, erythromycin, gentamicin, neomycin, tobramycin, besifloxacin, ciprofloxacin, gatifloxacin, levofloxacin, moxifloxacin, ofloxacin, bacitracin, chloramphenicol, gramicidin, natamycin, polymyxin B, sulfacetamide, tetracycline, trimethoprim, vancomycin, dexamethasone, difluprednate, fluorometholone, loteprednol, prednisolone, rimexolone, cyclosporine A, an NLRP3 inhibitor, diclofenac, ketorolac, bromfenac, nepafenac, flurbiprofen, lifitegrast, or a pharmaceutically acceptable salt, analogue, or derivative thereof.

5. The product combination of claim 4, wherein the NLRP3 inhibitor is Ac-YVAD-cmk, 2-APB, arglabin, BAPTA, BAY 11-7082, β-hydroxybutyrate (BHB), C172, CY-09, flufenamic acid, glybenclamide, INF39, isoliquiritigenin, MCC950, mefenamic acid, 3,4-methylenedioxy-β-nitrostyrene (MNS), OLT1177, oridonin, parthenolide, resveratrol, sulforaphane, tranilast, VX-765, or Z-VAD-FMK.

6. The product combination of any one of claims 1-5, wherein the additional therapeutic agent is present at a concentration of 0.001% to 5% w/v.

7. The product combination of any one of claims 1-6, wherein the one or more RNase comprises ranpirnase at a concentration of about 0.001% to about 1% w/v and one or more additional therapeutic agent comprises naphazoline, oxymetazoline, or brimonidine at a concentration of 0.001% to 0.1% w/v.

8. The product combination of any one of claims 1-7, comprising ranpirnase present in an amount of about 0.03% w/v and oxymetazoline present in an amount of about 0.01% to about 0.025% w/v.

9. The product combination of any one of claims 1-7, comprising ranpirnase present in an amount of about 0.03% w/v and brimonidine present in an amount of about 0.01% to about 0.025% w/v.

10. The product combination of any one of claims 1-9, wherein the ocular infection is viral conjunctivitis.

11. The product combination of claim 10, wherein the viral conjunctivitis is epidemic keratoconjunctivitis, pharyngoconjunctival fever, nonspecific sporadic follicular conjunctivitis, or chronic papillary conjunctivitis.

12. The product combination of any one of claims 10-11, wherein the viral conjunctivitis is caused by a virus infection from the Adenoviridae or Herpesviridae family.

13. The product combination of claim 12, wherein the virus infection is caused by Human adenovirus B, a Human adenovirus D, a Human adenovirus E, herpes simplex virus (HSV), varicella zoster virus (VZV), Epstein-Barr virus (EBV), human cytomegalovirus (CMV), or herpes zoster virus (HZV).

14. The product combination of claim 13, wherein the Human adenovirus B is a Human adenovirus B serotype 3, a Human adenovirus B serotype 7, a Human adenovirus B serotype 11, or any combination thereof.

15. The product combination of claim 13, wherein the Human adenovirus D is a Human adenovirus D serotype 8, a Human adenovirus D serotype 13, a Human adenovirus D serotype 19, a Human adenovirus D serotype 37, or any combination thereof.

16. The product combination of claim 13, wherein the Human adenovirus E is a Human adenovirus E serotype 4.

17. The product combination of any one of claims 1-16, wherein the product combination further comprises one or more pharmaceutically acceptable carriers and optionally one or more pharmaceutically acceptable components.

18. The product combination of any one of claims 1-17, wherein the one or more RNase and the one or more additional therapeutic agent are formulated in a single formulation.

19. The product combination of any one of claims 1-17, wherein the one or more RNase is in a first composition and the one or more additional therapeutic agent is in a second composition, and wherein the first composition is separate from the second composition.

20. The product combination of any one of claims 1-19, wherein the product combination is formulated as an ophthalmic formulation for use in an ophthalmic route of administration.

21. The product combination of any one of claims 1-20, wherein the product combination is formulated for administration by ocular instillation, ocular irrigation, intraocular injection, intracorneal injection, intravitreal injection, or subconjunctival injection.

22. The product combination of any one of claims 1-21, wherein the product combination is a controlled release delivery platform.

23. The product combination of claim 22, wherein the controlled release delivery platform is an extended-release formulation or a sustained release formulation.

24. The product combination of any one of claims 1-23, wherein the product combination is an ocular implant, an ophthalmic implant, a punctal plug, an intraocular implant, an intracorneal implant, or a subconjunctival implant.

25. The product combination of any one of claims 1-24, wherein the product combination comprises ranpirnase in an amount of about 25 μM and oxymetazoline in an amount of 0.01% to 0.025% w/v.

26. A method of reducing or inhibiting an ocular infection in a subject, the method comprising:
selecting a subject in need of reduction or inhibition of an ocular infection; and
administering to the subject:
a therapeutically effective amount of one or more ribonuclease (RNase); and
a therapeutically effective amount of one or more additional therapeutic agent, wherein the additional therapeutic agent is a vasoconstrictor, an antibiotic, an immunomodulatory compound, a steroid, or a combination thereof.

27. The method of claim 26, wherein the one or more RNase is ranpirnase, an analogue, variant, derivative, or fragment thereof.

28. The method of any one of claims 26-27, wherein the one or more additional therapeutic agent is naphazoline, tetrahydrozoline, phenylephrine, oxymetazoline, brimonidine, apraclonidine, ephedrine, azithromycin, erythromycin, gentamicin, neomycin, tobramycin, besifloxacin, ciprofloxacin, gatifloxacin, levofloxacin, moxifloxacin, ofloxacin, bacitracin, chloramphenicol, gramicidin, natamycin, polymyxin B, sulfacetamide, tetracycline, trimethoprim, vancomycin, dexamethasone, difluprednate, fluorometholone, loteprednol, prednisolone, rimexolone, cyclosporine A, an NLRP3 inhibitor, diclofenac, ketorolac, bromfenac, nepafenac, flurbiprofen, lifitegrast, or a pharmaceutically acceptable salt, analogue, or derivative thereof.

29. The method of claim 28, wherein the NLRP3 inhibitor is Ac-YVAD-cmk, 2-APB, arglabin, BAPTA, BAY 11-7082, β-hydroxybutyrate (BHB), C172, CY-09, flufenamic acid, glybenclamide, INF39, isoliquiritigenin, MCC950, mefenamic acid, 3,4-methylenedioxy-β-nitrostyrene (MNS), OLT1177, oridonin, parthenolide, resveratrol, sulforaphane, tranilast, VX-765, or Z-VAD-FMK.

30. The method of any one of claims 26-29, wherein the administering comprises instilling an ophthalmic composition in each eye of the subject four times daily, wherein the ophthalmic composition comprises ranpirnase in an amount of about 0.03% w/v and oxymetazoline in an amount of about 0.01% to about 0.025% w/v.

31. The method of any one of claims 26-29, wherein the administering comprises instilling an ophthalmic composition in each eye of the subject four times daily, wherein the ophthalmic composition comprises ranpirnase in an amount of about 0.03% w/v and brimonidine in an amount of about 0.01% to about 0.025% w/v.

32. The method of any one of claims 26-31, wherein the method inhibits or delays the ocular infection or prevents spread of the ocular infection.

33. The method of any one of claims 26-32, wherein the ocular infection is a viral conjunctivitis.

34. The method of claim 33, wherein the viral conjunctivitis is epidemic keratoconjunctivitis, pharyngoconjunctival fever, nonspecific sporadic follicular conjunctivitis, chronic papillary conjunctivitis, or herpetic conjunctivitis.

35. The method of any one of claims 26-34, wherein the administration is ophthalmic.

36. The method of any one of claims 26-35, wherein the one or more RNase is prepared in a first composition and the one or more additional therapeutic agent is prepared in a second composition, and wherein the first composition is administered prior to, concomitantly with, or subsequent to administration of the second composition.

37. The method of any one of claims 26-36, wherein the administering is two times a day.

38. The method of any one of claims 26-36, wherein the administering is four times a day.

39. The method of any one of claims 26-36, wherein the administering is eight times a day.

40. Use of a product combination in the manufacture of a medicament for the treatment of an ocular infection, the product combination comprising:
a therapeutically effective amount of one or more ribonuclease (RNase); and
a therapeutically effective amount of one or more additional therapeutic agent, wherein the additional therapeutic agent is a vasoconstrictor, an antibiotic, an immunomodulatory compound, a steroid, or a combination thereof.

41. The use of claim 40, wherein the one or more RNase is ranpirnase, an analogue, variant, derivative, or fragment thereof.

42. The use of any one of claims 40-41, wherein the one or more additional therapeutic agent is naphazoline, tetrahydrozoline, phenylephrine, oxymetazoline, brimonidine, apraclonidine, ephedrine, azithromycin, erythromycin, gentamicin, neomycin, tobramycin, besifloxacin, ciprofloxacin, gatifloxacin, levofloxacin, moxifloxacin, ofloxacin, bacitracin, chloramphenicol, gramicidin, natamycin, polymyxin B, sulfacetamide, tetracycline, trimethoprim, vancomycin, dexamethasone, difluprednate, fluorometholone, loteprednol, prednisolone, rimexolone, cyclosporine A, an NLRP3 inhibitor, diclofenac, ketorolac, bromfenac, nepafenac, flurbiprofen, lifitegrast, or a pharmaceutically acceptable salt, analogue, or derivative thereof.

43. The use of claim 42, wherein the NLRP3 inhibitor is Ac-YVAD-cmk, 2-APB, arglabin, BAPTA, BAY 11-7082, β-hydroxybutyrate (BHB), C172, CY-09, flufenamic acid, glybenclamide, INF39, isoliquiritigenin, MCC950, mefenamic acid, 3,4-methylenedioxy-β-nitrostyrene (MNS), OLT1177, oridonin, parthenolide, resveratrol, sulforaphane, tranilast, VX-765, or Z-VAD-FMK.

44. The use of any one of claims 40-43, wherein the product combination comprises ranpirnase present in an amount of about 0.03% w/v and oxymetazoline present in an amount of about 0.01% to about 0.025% w/v.

45. The use of any one of claims 40-43, wherein the product combination comprises ranpirnase present in an amount of about 0.03% w/v and brimonidine present in an amount of about 0.01% to about 0.025% w/v.

46. The use of any one of claims 40-45, wherein said medicament inhibits or delays the ocular infection.

47. The use of any one of claims 40-46, wherein the ocular infection is a viral conjunctivitis.

48. The use of claim 47, wherein the viral conjunctivitis is epidemic keratoconjunctivitis, pharyngoconjunctival fever, nonspecific sporadic follicular conjunctivitis, or chronic papillary conjunctivitis.

49. The use of any one of claims 40-48, wherein the medicament is formulated for ophthalmic administration.
